(19) United States
(12) Patent Application Publication
(10) Pub. No.: US 2017/0029892 A1
(43) Pub. Date: Feb. 2, 2017

(54) METHODS AND COMPOSITIONS FOR THE TREATMENT OF POST-TRAUMATIC STRESS DISORDER

(71) Applicant: Jay L. LOMBARD, New City, NY (US)

(72) Inventor: Jay L. LOMBARD, New City, NY (US)

(21) Appl. No.: 15/290,961

(22) Filed: Oct. 11, 2016

Related U.S. Application Data
(63) Continuation-in-part of application No. 13/937,168, filed on Jul. 8, 2013.
Application No. 13/937,168 is a continuation-in-part of application No. 13/210,808, filed on Aug. 16, 2011, now abandoned.
Application No. 13/937,168 is a continuation-in-part of application No. 13/365,076, filed on Feb. 2, 2012, now abandoned.
Application No. 13/937,168 is a continuation-in-part of application No. 13/739,970, filed on Jan. 11, 2013, now Pat. No. 8,706,526.
Application No. 13/739,970 is a continuation of application No. 13/371,227, filed on Feb. 10, 2012, now Pat. No. 8,355,927.
Application No. 13/371,227 is a continuation-in-part of application No. 13/290,603, filed on Nov. 7, 2011, now abandoned.
Application No. 13/290,603 is a continuation-in-part of application No. 14/324,524, filed on Jul. 7, 2014, now abandoned.
Application No. 14/324,524 is a continuation of application No. 13/074,967, filed on Mar. 29, 2011, now abandoned.
Application No. 13/074,967 is a continuation-in-part of application No. 12/790,262, filed on May 28, 2010, now abandoned.
(60) Provisional application No. 61/669,423, filed on Jul. 9, 2012.
(60) Provisional application No. 61/674,240, filed on Jul. 20, 2012.
(60) Provisional application No. 61/674,247, filed on Jul. 20, 2012.
(60) Provisional application No. 61/693,740, filed on Aug. 27, 2012.
(60) Provisional application No. 61/705,100, filed on Sep. 24, 2012.
(60) Provisional application No. 61/705,095, filed on Sep. 24, 2012.
(60) Provisional application No. 61/841,902, filed on Jul. 1, 2013.
(60) Provisional application No. 61/374,225, filed on Aug. 16, 2010.
(60) Provisional application No. 61/438,924, filed on Feb. 2, 2011.
(60) Provisional application No. 61/410,523, filed on Nov. 5, 2010.
(60) Provisional application No. 61/528,583, filed on Aug. 29, 2011.
(60) Provisional application No. 61/217,338, filed on May 29, 2009.
(60) Provisional application No. 61/325,098, filed on Apr. 16, 2010.
(60) Provisional application No. 61/410,523, filed on Nov. 5, 2010.
(60) Provisional application No. 61/321,065, filed on Apr. 5, 2010.
(60) Provisional application No. 61/321,281, filed on Apr. 6, 2010.

(51) Int. Cl. C12Q 1/68; A61K 31/4184; A61K 31/65; A61K 31/4178

(52) U.S. Cl. CPC C12Q 1/6883; A61K 31/4178; A61K 31/4184; A61K 31/65; C12Q 2600/156; C12Q 2600/154; C12Q 2600/106

(57) ABSTRACT

Methods and compositions are disclosed to treat neuropsychiatric disorders post-traumatic stress disorder (PTSD). In particular, described herein are angiotensin receptor blockers (ARBs), and in particular the combination of one or more ARB (such as telmisartan) and an agent that enhances the delivery of the ARB across the blood-brain barrier (such as minocycline). PTSD may be treated using a combination of telmisartan and minocycline at levels of each that are, by themselves, infective to treat PTSD. Also described herein are methods for treating PTSD by first identifying patents for whom the use of an ARB treatment would be effective, by determining that patient has a dysfunction in their angiotensin converting enzyme and/or other genes in the autonomic arousal axis.

### CROSS REFERENCE TO RELATED APPLICATIONS

This patent application claims priority as a continuation-in-part of U.S. patent application Ser. No. 13/937,168, filed Jul. 8, 2013, titled “METHODS AND COMPOSITIONS FOR THE TREATMENT OF NEUROPSYCHIATRIC DISORDERS,” Publication No. US-2013-0295203-A1, which claims priority to the following provisional patent applications: U.S. Provisional Patent Application No. 61/669,423, filed Jul. 9, 2012, titled “MEDICAL FOODS FOR THE TREATMENT OF SUBTYPES OF AUTISM AND METHODS OF USE;” U.S. Provisional Patent Application No. 61/674,240, filed Jul. 20, 2012, titled “USE OF THE COMT ACTIVITY TO GUIDE TREATMENT OF SUBTYPES OF DEPRESSION AND CHRONIC PAIN SYNDROMES;” U.S. Provisional Patent Application No. 61/674,247, filed Jul. 20, 2012, titled “COMPANION DIAGNOSTIC-BASED MEDICAL FOOD FOR TREATMENT OR PREVENTION OF DEMENTIA;” U.S. Provisional Patent Application No. 61/693,740, filed Aug. 27, 2012, titled “COMPANION DIAGNOSTICS AND MEDICAL FOOD INTERVENTIONS IN NEUROPSYCHIATRY;” U.S. Provisional Patent Application No. 61/705,100, filed Sep. 24, 2012, titled “COMPANION DIAGNOSTICS AND MEDICAL FOOD INTERVENTIONS IN NEUROPSYCHIATRY;” U.S. Provisional Patent Application No. 61/705,095, filed Sep. 24, 2012, titled “SIALIC ACID COMPOUNDS FOR THE PREVENTION OR TREATMENT OF AUTISM AND PERVASIVE DEVELOPMENTAL DELAY;” and U.S. Provisional Patent Application No. 61/841,902, filed Jul. 1, 2013, titled “METHODS AND COMPOSITIONS FOR THE TREATMENT OF NEUROPSYCHIATRIC DISORDERS,” each of which is herein incorporated by reference in its entirety.

U.S. patent application Ser. No. 13/937,168 also claims priority as a continuation-in-part of U.S. patent application Ser. No. 13/210,808, filed Aug. 16, 2011 and titled “MEDICAL FOODS FOR THE TREATMENT OF DEVELOPMENTALLY-BASED NEUROPSYCHIATRIC DISORDERS VIA MODULATION OF BRAIN GLYCINE AND GLUTATHIONE PATHWAYS,” Publication No. US-2012-0041066-A1, now abandoned, which claims priority to U.S. Provisional Patent Application No. 61/374,225, filed Aug. 16, 2010, titled “MEDICAL FOODS FOR THE TREATMENT OF DEVELOPMENTALLY-BASED NEUROPSYCHIATRIC DISORDERS VIA MODULATION OF BRAIN GLYCINE AND GLUTATHIONE PATHWAYS,” each of which is herein incorporated by reference in its entirety.

U.S. patent application Ser. No. 13/937,168 also claims priority as a continuation-in-part of U.S. patent application Ser. No. 13/365,076, filed Feb. 2, 2012 and titled “DIAGNOSIS AND TREATMENT OF THE PRODROMAL SCHIZOPHRENIC STATE,” Publication No. US-2012-0195984-A1, now abandoned, which claims priority to U.S. Provisional Patent Application No. 61/438,924, filed Feb. 2, 2011, titled “TREATMENT OF THE PRODROMAL SCHIZOPHRENIC STATE,” each of which is herein incorporated by reference in its entirety.

U.S. patent application Ser. No. 13/937,168 also claims priority as a continuation-in-part of U.S. patent application Ser. No. 13/739,970, filed Jan. 11, 2013, titled “NEUROPSYCHIATRIC TEST REPORTS,” now U.S. Pat. No. 8,706,526, which claims priority as a continuation of U.S. patent application Ser. No. 13/371,227, filed Feb. 10, 2012, titled “NEUROPSYCHIATRIC TEST REPORTS,” now U.S. Pat. No. 8,355,927, which claims priority as a continuation-in-part of U.S. patent application Ser. No. 13/290,603, filed Nov. 7, 2011, titled “NEUROPSYCHIATRIC TEST REPORTS,” Publication No. US-2012-0115147-A1, now abandoned, which claims priority to U.S. Provisional Patent Application No. 61/410,523, filed Nov. 5, 2010, titled “TREATMENT RESISTANT DEPRESSION DIAGNOSTIC TEST REPORT,” each of which is herein incorporated by reference in its entirety.

U.S. patent application Ser. No. 13/371,227 also claims priority to U.S. Provisional Patent Application No. 61/528,583, filed Aug. 29, 2011 and titled “INTERPRETIVE BIOMARKER SCREENING REPORTS FOR DIAGNOSIS AND TREATMENT OF PSYCHIATRIC DISORDERS,” each of which is herein incorporated by reference in its entirety.

This patent application also claims priority as a continuation-in-part of U.S. patent application Ser. No. 14/324,524, filed Jul. 7, 2014, titled “METHODS FOR ASSESSMENT AND TREATMENT OF MOOD DISORDERS VIA SINGLE NUCLEOTIDE POLYMORPHISMS ANALYSIS,” Publication No. US-2014-0323424-A1, which claims priority as a continuation of U.S. patent application Ser. No. 13/074,967, filed Mar. 29, 2011, titled “METHODS FOR ASSESSMENT AND TREATMENT OF MOOD DISORDERS VIS SINGLE NUCLEOTIDE POLYMORPHISMS ANALYSIS,” Publication No. 2011-0237537-A1, now abandoned which claims priority as a continuation-in-part of U.S. patent application Ser. No. 12/790,262, filed May 28, 2010, titled “METHOD FOR ASSESSMENT AND TREATMENT OF DEPRESSION VIA UTILIZATION OF SINGLE NUCLEOTIDE POLYMORPHISMS ANALYSIS,” Publication No. 2010-0304371-A1, now abandoned, which claims priority to U.S. Provisional Patent Application No. 61/217,338, filed May 29, 2009, titled “SYSTEM AND METHOD FOR DIAGNOSIS AND TREATMENT OF COMMON MENTAL HEALTH COMPLAINTS” and U.S. Provisional Patent Application 61/325,098, filed Apr. 16, 2010, titled “MODULATION OF SEROTONIN REUPTAKE BASED ON GENOTYPE TO TREAT DEPRESSION,” each of which is herein incorporated by reference in its entirety.

U.S. patent application Ser. No. 13/074,967 also claims priority to U.S. Provisional Patent Application No. 61/410,523, filed Nov. 5, 2010, titled “TREATMENT RESISTANT DEPRESSION DIAGNOSTIC TEST REPORT;” 61/321,065, filed Apr. 5, 2010, titled “MEDICAL FOODS FOR THE TREATMENT OF DEPRESSION AND NEURODEGENERATIVE DISORDERS;” and 61/321,281, filed Apr. 6, 2010, titled “TREATMENT OF ALZHEIMER'S DISEASE BY MODULATION OF ANTIMICROBIAL PEPTIDES,” each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Personalized medicine for neuropsychiatric disorders, in particular, post-traumatic stress disorder (PTSD).

### BACKGROUND

In the last fifty years, a tremendous amount of research has begun to elucidate the causes, characteristics and treatments of many neuropsychiatric disorders. Unfortunately, this process has not efficiently translated into effective patient treatments. The term “treatment resistance” is more the norm than the exception in psychiatry. The problem of treatment resistance in psychiatry, the lack of biomarkers for diagnosis, the fact of similar symptoms in different categorical diagnoses, and the difficulty in drawing boundaries between disorders, all necessitates a paradigm shift from categorical to dimensional diagnostics.

Virtually all brain disorders may cause psychiatric symptoms. The term “neuropsychiatric disorders” may refer to brain disease or dysfunction that causes psychiatric symptoms. Examples of neuropsychiatric disorders include depression (including treatment resistant depression, bipolar depression, etc.), schizophrenia, post-traumatic stress disorder (PTSD) and other anxiety disorders, autism, ADHD, and the like. Even neurodegenerative disorders, such as dementia may present with primary psychiatric symptoms, especially in the earlier stages of the disease. As another example, the significant co morbidity of PTSD and closed head injuries (e.g., traumatic brain injury or TBI), especially in veterans, may present with primary psychiatric symptomatology.

Although various research and clinical studies have looked for diagnostic and therapeutic indicators in an almost overwhelming variety of genomic markers, gene expression markers and protein markers, this vast and growing body of data has proven difficult to interpret. Most physicians are unable to synthesize the tremendous amount of information on possible risk factors and indicators in order to apply this information clinically to diagnose and/or treat patients.

Thus, there is an as yet unmet need for reports, panels and/or kits that would allow a medical professional to apply the most relevant genetic, epigenetic, transcriptomic, proteomic and functional imaging tests in a meaningful manner to their patients.

Genes associated with neurotransmitters, ionic channels (calcium, sodium and potassium) and metabolic pathways (immune and inflammatory), have been found to be abnormal in patients with various neuropsychiatric disorders. For instance, genes which regulate serotonin pathways, including genes coding for receptors, metabolism and reuptake mechanisms, are associated with mood disturbances. Furthermore, other genetic-neurotransmitter pathways, including dopamine, norepinephrine and glutamate may be associated with depression or risk of dementia. Regarding ion channels, pathological states in the brain can result from changes in which alter membrane excitability. Phenomenologically, alterations in ion channels may be seen clinically as paroxysmal, recurrent, or intermittent disturbances. Genes related to cerebral metabolism, such as methylation and the like, also impart changes with neuropsychiatric implications. For example, genes related to oxidation, mitochondrial function, proteasomal degradation and insulin and its associated second messenger systems (gene pathways) may also have neuropsychiatric implications. Unfortunately, what is not well-understood is how to apply such genetic or expression-related information to patient treatment in a robust and useful manner, particularly for neuropsychiatric disorders. Genes that regulate immune processes are also relevant in clinical assessments as variants in glial cell activity have been associated with depression, schizophrenia, bipolar disease and dementia.

By analyzing disorders using a spectrum of biomarkers, such as SNP-based gene analysis, subtypes of neuropsychiatric conditions can be differentiated and treated in a personalized manner. This analysis may allow a deeper understanding of a patient's health across a variety of neuropsychiatric categories. Further, the employment of such analysis will allow mental health professionals to treat individuals with more specific and targeted interventions. Therefore, the approach described herein may be used to reveal genomic influences on trait components of a variety of neuropsychiatric disorders (regardless of categorical classification) and may help identify subpopulations of patients that can benefit from more targeted pharmacotherapy.

This approach has was described in U.S. patent application Ser. No. 13/371,227 (“the '227 application” now U.S. Pat. No. 8,355,927), which this application claims priority as a continuation-in-part of The '227 application, as well as subsequent family member, including U.S. patent application Ser. No. 13/937,168, to which this application also claims priority as a continuation-in-part, identified PTSD in particular, as a neuropsychiatric disorder that may be related to a dysfunction in the patient's angiotensin converting enzyme and/or other genes in the autonomic arousal axis, and for the first time proposed that an effective treatment may be angiotensin receptor blockers (“ARBs” such as Candesartan and the like).

PTSD is a debilitating neuropsychiatric disorder typically induced by exposure to a severe trauma and is associated with functional impairments and increased physical and mental health risks. PTSD patients may have a six-fold higher risk of suicide than non-PTSD patients. The treatment of PTSD is challenging, and may include many years of individual and group therapy and treatment with medications such as conventional antidepressants, anxiolytic drugs, β-adrenergic antagonists, opiates, or Cortisol with variable results. PTSD is a devastating and complex pathological anxiety condition that is characterized by severe distress and impairment in mental and physical functioning. Symptoms typically include intense anxiety, hyper-arousal, flashbacks and sleep disturbances. The impact and consequences for individuals diagnosed with PTSD include depression, substance abuse, violence, inability to maintain intimate relationships, suicide and premature mortality, and possibly risk of dementia as well. PTSD with co morbid depression is especially common and problematic and requires novel approaches because there are significant rates of treatment resistance and failures. PTSD is also a public health dilemma because nearly 80% of residents experience traumatic events in their lifetime. Given the predominance of PTSD and its negative consequences to long term health, it is very important to identify measurable and quantifiable biological parameters that may indicate disturbances of the blood brain barrier that may impair treatment response as well as be reflective of the primary pathophysiology as well.

Although numerous drugs exist in the market today to treat the symptoms or manage the progression of these diseases, most have modest or limited efficacy. Frequently, polypharmacy is employed to optimize therapy to the specific needs of patients at different stages of the disease. Current guidelines for the treatment of PTSD recommend initiation of a selective serotonin reuptake inhibitor or serotonin-norepinephrine reuptake inhibitor (SNRI, e.g., venlafaxine). If the initial trial of an SSRI or SNRI is not effective after 4 to 8 weeks, consideration of another first-line SSRI/SNRI or mirtazapine is warranted. A recent meta-analysis showed that the effect sizes for pharmacological treatments for PTSD are very low, with less than 30% of patients achieving benefit. Prolonged Exposure (PE) and Cognitive Processing Therapy target avoidance, now considered by DSM-5 to be essential for diagnosis. However, dropout rates are high in trauma-focused treatments and are attributed to the initial increase in symptoms as patients begin describing the trauma memory, which activates an avoidance mechanism (i.e., cancelling or no-showing for subsequent appointments).

Both PTSD and traumatic brain injury (TBI) commonly occur in the general population, both share some pathophysiological characteristics and both are associated with cognitive impairment and sleep disruption. PTSD and TBI present with a number of overlapping symptoms, which can lead to over-diagnosis or misdiagnosis. Both conditions are associated with co-morbidities important in diagnosis and treatment planning. Both disorders are common in Iraq and Afghanistan veterans, and together they have been termed the “signature wounds” of these conflicts. TBI has emerged as a clear and important risk factor for the development of PTSD, substantial overlap in symptoms of PTSD and postconcussive symptoms may result in considerable diagnostic confusion and treatment of individuals with comorbid PTSD and TBI may present special challenges, and yet there is currently very little evidence base to guide pharmacologic and psychotherapeutic treatment in this population.

There is a need for effective treatments for diagnosing and treating patients having or likely to have PTDS.

Although ARBs have been traditionally used to treat vascular disorders such coronary artery disease, heart failure, high blood pressure, or kidney disease. These drugs have not been widely used to treat neuropsychiatric disorders, and particularly not PTDS. This may be due, at least in part, to the difficulty in getting these drugs (examples of ARBs may include candesartan, eprosartan, irbesartan, losartan, olmesartan, telmisartan, and valsartan).

Although various research and clinical studies have looked for diagnostic and therapeutic indicators to refine diagnosis and treatment, in an almost overwhelming variety of genomic markers, gene expression markers and protein markers, this vast and growing body of data has proven difficult to interpret. Most physicians are unable to synthesize the tremendous amount of information on possible risk factors and indicators in order to apply this information clinically to diagnose and/or treat patients. Thus, there is an as yet unmet need for reports, panels and/or kits that would allow a medical professional to apply the most relevant genetic, epigenetic, transcriptomic, proteomic and functional imaging tests in a meaningful manner to their patients. It is also critical to provide a proper analysis that allows the medical profession to understand and interpret the results of such tests, as well as have a resource to call upon for clarification of their interpretations.

Described herein are methods of identifying (e.g., by genetic screening) neuropsychiatric patients, and particularly patients having or likely to have PTSD, that may respond to a drug treatment including one or more ARB, as well as compositions including one or more ARB that may be used to effectively treat PTSD.

### SUMMARY OF THE DISCLOSURE

Described herein are methods to identify and/or treat post-traumatic stress disorder (PTSD), as well as compositions, typically including an angiotensin receptor blocker (ARB) that are surprisingly and particularly effective in treating PTSD.

In some variations, the methods described herein include methods for determining if a patient will be likely to respond to an ARB drug therapy by examining the patient's autonomic arousal axis, and in particular the genes encoding proteins that form part of the patient's autonomic arousal axis. The autonomic arousal axis functionally relates to stress and autonomic hyperactivity. The functional brain circuits involved typically include the amygdala and hypothalamus. Patients with disturbances in this axis may display recognizable clusters of symptoms including heightened arousal, panic, intractable anxiety, PTSD and the like. The principle neurotransmitter pathways implicated for this area include the serotonin/norepinephrine neurotransmitter and corticotrophin/angiotensin pathways. Dysregulation of these pathways may result in problems of autonomic arousal. By example, genes that are part of larger gene families and one or more pathways may include, but are not limited to: serotonin transporter (SLC6A4 or SERT or 5-HTT or 5-HTTLPR), FK506-binding protein 5 (FKBP5), serotonin 5-HT-1A receptor (HTR1A or 5-HT1A), angiotensin-converting-enzyme (ACE), Neuropeptide Y (NPY), catechol-O-methyltransferase (COMT), and the like. Abnormal biomarker detection would indicate dysfunction in this axis with clinically associated disturbances in emotional vigilance, anxiety, panic, PTSD and the like. Potential treatments for this endophenotype, based upon identification of these biomarkers, may involve angiotensin receptor blockers (such as Candesartan and the like) which may reduce autonomic hyperactivity.

Also described are compositions including one or more ARB and methods of using them to treat PTSD. Although it may be beneficial to use these compositions in patients for whom a dysfunction in the autonomic arousal axis (e.g., one or more genes such as ACE and FKBP5) have been identified, these compositions may be also be beneficially used in patients even without first identifying such potential genetic dysfunction (including a polymorphism in one or more of the genes of this pathway).

The inventor has surprisingly discovered that certain types of therapeutic agents can be used in combination to treat PTSD and/or TBI. For example, described herein are pharmaceutical compositions and methods of preparing and using them (e.g., prescribed and self-administered) to treat a patient. These compositions, which typically include an ARB, and in particular, may include the ARB telmisartan (2-(4-{[4-Methyl-6-(1-methyl-1H-1,3-benzodiazol-2-yl)-2-propyl-1H-1,3-benzodiazol-1-yl]methyl}phenyl)benzoic acid). Although ARBs may be used by themselves to treat PTDS as described in one or more of the parent applications to which this patent claims priority (see, e.g., U.S. Pat. No. 8,355,927), the compositions described herein may include an additional active ingredient that these one or more ARBs may be used with such as the tetracycline antibiotic minocycline ((2E,4S,4aR,5aS,12aR)-2-(amino-hydroxy-methylidene)-4,7-bis(dimethylamino)-10,11,12a-trihydroxy-4a,5,5a,6-tetrahydro-4H-tetracene-1,3,12-trione); the combination of these two active ingredients has been surprisingly found to be remarkable effective at concentrations at which neither active ingredient alone is effective in treating PTSD and/or TBI. These therapeutic agents may be formulated in a single preparation (e.g., a single tablet, capsule, or the like, which may be designed to produce a sustained and/or controlled release) and administered orally. Alternatively or additionally, these agents may be administered concurrently but separately and/or may be administered in non-oral forms (e.g., intravenously).

Regardless of the precise formulation or configuration, the compositions described herein can include at least one active ingredient that targets the hypothalamo-pituitary-adrenal (HPA) axis and in particularly the angiotensin receptors, and at least one active ingredient that targets microglial cells involved in central nervous system mediated immune imbalances. Specifically, the first agent may be an ARB such as candesartan or telmisartan, which are commonly used to treat cardiovascular diseases. The second agent may be minocycline, which is used to treat infections. Both of these active agents/ingredients (drugs), when used in the formulations described herein, may act synergistically to achieve surprisingly better clinical outcomes and may address abnormal HPA axis activity, immune dysregulation and alterations in the blood brain barrier which otherwise may not be addressed through current approaches in treating these disorders.

For example, described herein are methods of treating a patient having post-traumatic stress disorder (PTSD) (either with or without co-morbid TBI), as schematically illustrated in FIG. 1. For example, the method may include: determining that the patient has a polymorphism in the patient's angiotensin converting enzyme (ACE) gene 101; treating the patient with an angiotensin receptor blocker 103.

The angiotensin receptor blocker may be one or more of losartan or telmisartan; in particular, the ARB may be telmisartan. Treating the patient with an angiotensin receptor blocker may comprise treating the patient with an angiotensin receptor blocker and a tetracycline, in particular, minocycline 105. For example, treating the patient with an angiotensin receptor blocker may comprise treating the patient with an angiotensin receptor blocker (ARB) and a tetracycline, wherein the ARB comprises telmisartan and the tetracycline comprises minocycline.

The amount by mass of the first active agent (e.g., telmisartan) may typically be greater than the amount by mass of the second active agent (e.g., minocycline). For example, the ratio of telmisartan to minocycline may be between 100:1 and 1:1 (e.g., about: 100:1, 90:1, 80:1, 75:1, 70:1, 65:1, 60:1, 55:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1). For example, treating may comprise giving the patient a dose form comprises telmisartan between about 10-100 mg of telmisartan and about 10-400 mg of minocycline a day. The dose may be a single daily dose or a multiple doses spread out throughout the day. The dose may be a combination dose in which the telmisartan and minocycline are formulated together (along with inactive ingredients) to be delivered together. Both the telmisartan and/or the minocycline may be formulated as a salt form, and/or may be modified (e.g., conjugated), co-distributed, or otherwise formulated for concurrent (in time) release, etc. 107.

For example, treating may comprise giving the patient a single combination dose form comprises telmisartan between about 10-100 mg of telmisartan and about 50-200 mg of minocycline per day.

In methods in which it is determined that the patient has a polymorphism in the ACE gene, the polymorphism may be, e.g., the patient is CC homozygous in rs4311. Other polymorphism affecting the function of the ACE gene may also be used to confirm that the patient is particularly likely to respond to treatment with an ARB or ARB formulation as described herein. One or more other genetic markers may be reviewed and used to guide treatment. For example, any of these methods may include determining that the patient has a dysfunction in the FK506 binding protein (FKBP) gene, and/or a dysfunction in the Multi-Drug Resistance 1 (MDR1) gene.

For example, a method of treating a patient having post-traumatic stress disorder (PTSD) may include: determining that the patient has a polymorphism in the patient's angiotensin converting enzyme (ACE) gene; treating the patient with one or more of: an angiotensin-converting enzyme inhibitor and angiotensin receptor blocker if the polymorphism indicates a dysfunction in the ACE gene.

As mentioned above, it may not be necessary to identify the genetic background of the individual before treating PTSD and/or TBI using any of the ARBs and ARB formulations described herein. For example, also described herein are methods of treating a patient having post-traumatic stress disorder (PTSD) with/or without co-morbidity for TBI. This is schematically illustrated in FIG. 2. This method may include: delivering a first agent that targets the patient's hypothalamo-pituitary-adrenal (HPA) axis and down-regulates an effect of angiotensin in a subject to whom the composition is administered, wherein the first agent is telmisartan or a pharmaceutically acceptable salt thereof 201; concurrently delivering a second agent that targets pro-inflammatory microglial states wherein the second agent is minocycline or a pharmaceutically acceptable salt thereof 203; wherein both the first agent and the second agent are delivered in amounts that are ineffective to treat PTSD when either the first agent or the second agent is administered alone.

For example, both the first agent and the second agent may be delivered as part of a combination dose form 205 comprising telmisartan and minocycline. For example, both the first agent and the second agent may be delivered as part of a combination dose form comprising telmisartan and minocycline, wherein the combination dose form comprises 10-100 mg of telmisartan and 10-400 mg of minocycline. Both the first agent and the second agent may be delivered as part of a combination dose form comprising telmisartan and minocycline, wherein the combination dose form comprises 10-100 mg of telmisartan and 50-200 mg of minocycline 207. As mentioned, any of these methods method may also include determining that the patient has a polymorphism in the angiotensin converting enzyme (ACE) gene prior to delivering the first agent and the second agent. For example, the method may include determining that the patient has a polymorphism in the ACE gene comprises determining that the patient is CC homozygous in rs4311 prior to delivering the first agent and the second agent. Any of these methods may include determining that the patient has a dysfunction in the FK506 binding protein (FKBP) gene prior to delivering the first agent and the second agent, and/or the Multi-Drug Resistance 1 (MDR1) gene prior to delivering the first agent and the second agent.

Further, any of these methods may include monitoring the patient's response to the treatment by measuring a biomarker.

For example, described herein are methods of treating a patient having post-traumatic stress disorder (PTSD), the method comprising: delivering a first agent that targets the patient's hypothalamo-pituitary-adrenal (HPA) axis and down-regulates an effect of angiotensin in a subject to whom the composition is administered, wherein the first agent is telmisartan or a pharmaceutically acceptable salt thereof; concurrently delivering a second agent that targets pro-inflammatory microglial states wherein the second agent is minocycline or a pharmaceutically acceptable salt thereof; and wherein both the first agent and the second agent are delivered as part of a combination dose form comprising telmisartan and minocycline in amounts that are ineffective to treat PTSD when either the first agent or the second agent is administered alone, wherein the combination dose form comprises 10-100 mg of telmisartan and 10-400 mg of minocycline.

In general, pharmaceutical compositions for treating PTSD and/or TBI are also described herein. For example, pharmaceutical compositions for the treatment of post-traumatic stress disorder (PTSD) with or without co-morbid TBI are described herein. A pharmaceutical composition for the treatment of post-traumatic stress disorder (PTSD) with or without co-morbid TBI may include active agents of the composition consisting essentially of: telmisartan or a pharmaceutically acceptable salt thereof, and minocycline or a pharmaceutically acceptable salt thereof. Additional (non-essential) inactive ingredients may be included with any of these compositions, such as one or more of: a carrier, an excipient, a preservative, flavorant, colorant, etc. (including any inactive ingredients known in the art).

In any of these variations, the pharmaceutical composition may generally have about 10-100 mg of telmisartan and about 10-400 mg of minocycline. For example, the pharmaceutical composition may have about 10-100 mg of telmisartan and about 50-200 mg of minocycline.

A pharmaceutical composition for the treatment of post-traumatic stress disorder (PTSD) may include: a first agent that targets the hypothalamo-pituitary-adrenal (HPA) axis and down-regulates an effect of angiotensin in a subject to whom the composition is administered, wherein the first agent is telmisartan or a pharmaceutically acceptable salt thereof; and a second agent that targets pro-inflammatory microglial states wherein the second agent is minocycline or a pharmaceutically acceptable salt thereof, wherein said pharmaceutical composition is a single combination dosage form, and wherein both the first agent and the second agent are present within the composition in an amount that is ineffective to treat PTSD with or without co-morbid TBI when either the first agent or the second agent is administered alone because of reduced ability to overcome the blood brain barrier.

As mentioned above, the combination dose form may comprises between about 10-100 mg (or between about 1 mg and 400 mg, between about 10 mg and 300 mg, between about 10 mg and 150 mg, between about 10 mg and 120 mg, between about 10 mg and 100 mg, between about 20 mg and 400 mg, between about 20 mg and 300 mg, between about 20 mg and 200 mg, between about 30 mg and 400 mg, between about 30 mg and 300 mg, between about 30 mg and 200 mg, between about 30 mg and 100 mg, between about 50 mg and 400 mg, between about 50 mg and 300 mg, between about 50 mg and 200 mg, between about 50 mg and 100 mg, between about 10 mg and 90 mg, between about 10 mg and 80 mg, between about 10 mg and 70 mg, between about 10 mg and 60 mg, between about 10 mg and 50 mg, etc.) of telmisartan and about 10-400 mg of minocycline (e.g., between about 50-200 mg, between about 10-300 mg, between about 10-200 mg, between about 10-150 mg, between about 10-100 mg, between about 10-90 mg, between about 10-80 mg, between about 10-70, between about 10-60, between about 10-50 mg, between about 20-400 mg, between about 20-300, between about 20-200 mg, between about 20-100 mg, between about 20-90 mg, between about 30-500 mg, between about 30-400 mg, between about 30-300 mg, between about 30-200 mg, between about 30-100 mg, etc.; between about 40-500 mg, between about 40-400 mg, between about 40-300 mg, between about 40-200 mg, between about 40-100 mg, between about 50-500 mg, between about 50-400 mg, between about 50-300 mg, between about 50-100 mg, between about 50-80 mg, etc.). The ratio of the amount of the first agent to the second agent is between about 100:1 and 1:1, as mentioned above.

The pharmaceutical composition may include one or more additional agents, wherein the one or more additional agents comprise one or more of: a carrier, an excipient, a preservative, and a substance that increases the blood brain barrier permeability of one or more of the first agent and the second agent.

For example, a pharmaceutical composition for the treatment of post-traumatic stress disorder (PTSD) with or without co-morbid TBI may include: a first agent that targets the hypothalamo-pituitary-adrenal (HPA) axis and down-regulates the effect of angiotensin in a subject to whom the composition is administered, wherein the first agent is telmisartan or a pharmaceutically acceptable salt thereof; a second agent wherein the second agent is minocycline or a pharmaceutically acceptable salt thereof; and one or more agents, wherein the one or more agents comprises a carrier or excipient, a preservative, or a substance that increases the blood brain barrier permeability of the first agent or the second agent, wherein said pharmaceutical composition is a single combination dosage form, wherein both the first agent and the second agent are present within the composition in an amount that is ineffective to treat PTSD and/or TBI when either the first agent or the second agent is administered alone.

### BRIEF DESCRIPTION OF THE DRAWINGS

FIG. 1 is a schematic illustration of an exemplary method of treating a patient having post-traumatic stress disorder (PTSD).

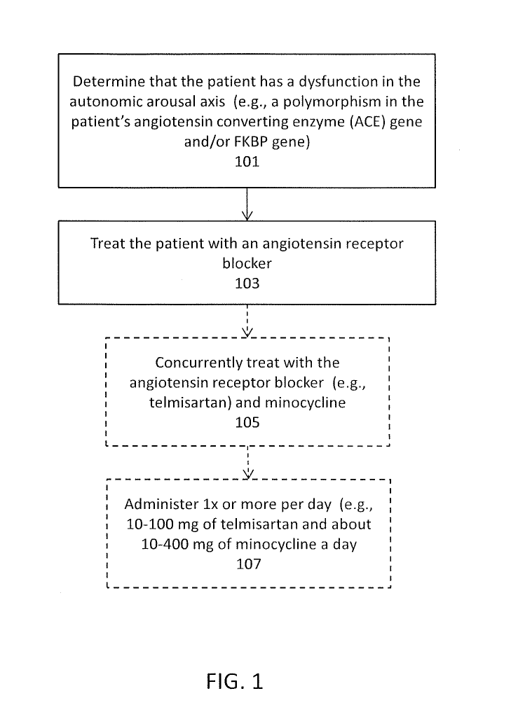

FIG. 2 is another schematic of a method of treating a patient having PTSD.

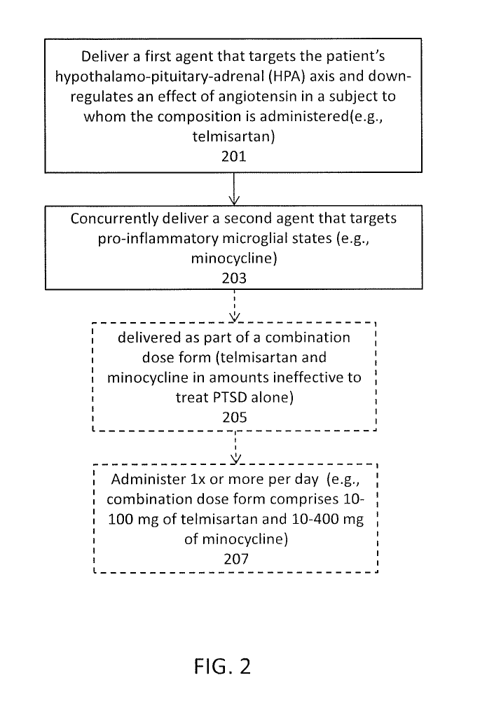

### DETAILED DESCRIPTION

In general, described herein are methods and compositions for treating post-traumatic stress disorder (PTSD) and/or traumatic brain injury (TBI). In particular, described herein are compositions including one or more angiotensin receptor blocker (ARB), including combination of one or more ARB (such as telmisartan) and an agent that enhances the delivery of the ARB across the blood-brain barrier (such as minocycline), to treat PTDS. The efficacy of any methods for treating PTSD using any of these compositions may be enhanced by first identifying patents for whom the use of an ARB treatment would be effective, for example, by determining that patient has a dysfunction in their angiotensin converting enzyme and/or other genes in the autonomic arousal axis (e.g., the hypothalamo-pituitary-adrenal (HPA) axis).

Part one of this disclosure below describes the identification of one or more dysfunctions in a patient's autonomic arousal axis, including kits and apparatuses for determining such dysfunctions, as well general methods of treating patients for whom a dysfunction has been identified. Part two of this disclosure describes formulations (pharmaceutical compositions) that may be particularly effective in treating such patients, either with or without a preliminary identification that the patient has a dysfunction in the autonomic arousal axis.

Part 1

Described herein are methods and apparatus for determining if a subject would benefit from any of the pharmaceutical compositions to treat PTSD described herein. In particular, describe herein are methods of performing a dimensional assay of a patient, including one or more biomarkers in at least the autonomic arousal axis, and possibly one or more additional axes. A biomarker may include a genetic marker, including a polymorphism, an epigenic marker (e.g., methylation) or other post-intervention biomarker, a behavior biomarker (phenotype), or the like. This method may be automated using a processor that is configured to perform the analysis and provide output including a description of one or more pharmaceutical composition appropriate for the subject.

Examples of methylation analysis as a “post intervention” biomarker can be anticipated as markers of epigenetics; the influence of a specific intervention on the methylation of a gene or group of genes.

A step of presenting a weighted index of confidence level for the interpretive analysis of a neuropsychiatric domain analysis may be summarized in the report in a key, or they may be self-qualifying (e.g., the index may indicate “high,” “medium” or “low” confidence values).

For example, described herein are methods and systems, including articles of manufacture such as reports, for guiding therapeutic treatment of neuropsychiatric disorders including in particular PTDS. For example, described herein are systems, methods and articles of manufacture relevant to treatment-resistant psychiatric disorders including PTSD.

In general, the systems described herein provide a dimensional, rather than categorical, assay, screen, test, and/or report relevant to treating neuropsychiatric disorders. The dimensional assays, and dimensional assay reports, described herein, typically use a collection or set of biomarkers that are relevant to one or more areas useful for understanding pathophysiological pathways of the brain underlying many classes of neuropsychiatric disorders. These areas may be defined or described based on the anatomical and/or functional biological relationships, which correlate with the dimensional symptom spectrum of a particular condition. Of particular interest are three areas, which are described in greater detail below: limbic based the autonomic arousal area (or axis) which includes the amygdala and hypothalamic-pituitary-adrenal (HPA) axis; the emotional valence and reward and the executive brain function area (axis), which includes the prefrontal cortex, anterior cingulate and nucleus accumbens; and the domain which mediates synaptic strength and abnormal brain circuitry, long-term potentiation and long-term depression axis.

The autonomic arousal area (or axis) functionally relates to stress and autonomic hyperactivity. The functional brain circuits involved typically include the amygdala and hypothalamus. Patients with disturbances in this axis may display recognizable clusters of symptoms including heightened arousal, panic, intractable anxiety, PTSD and the like. The principle neurotransmitter pathways implicated for this area include the serotonin/norepinephrine neurotransmitter and corticotrophin/angiotensin pathways. Dysregulation of these pathways may result in problems of autonomic arousal. By example, genes that are part of larger gene families and one or more pathways may include, but are not limited to: serotonin transporter (SLC6A4 or SERT or 5-HTT or 5-HTTLPR), FK506-binding protein 5 (FKBP5), serotonin 5-HT-1A receptor (HTR1A or 5-HT1A), angiotensin-converting-enzyme (ACE), Neuropeptide Y (NPY), catechol-O-methyltransferase (COMT), and the like. Abnormal biomarker detection would indicate dysfunction in this axis with clinically associated disturbances in emotional vigilance, anxiety, panic, PTSD and the like. Potential treatments for this endophenotype, based upon identification of these biomarkers, may involve noradrenergic modulators (such as NRIs), angiotensin receptor blockers (such as Candesartan and the like), lithium or other agents which reduce autonomic hyperactivity.

The emotional valence and reward/executive brain function area (axis) relates to the pain/pleasure response may involve the functional brain circuits of the prefrontal cortex, ventral striatum, and nucleus acumbens regions of the brain. Dimensionally, individuals with dysfunction in this axis may exhibit abnormalities in motivation, attention, cravings, addiction and the like. The principle neurotransmitter pathway implicated in this axis is dopamine (dopaminergic); dysregulation of dopamine neurotransmission in these regions may result in dysfunction of this axis. Representative gene markers that are part of larger gene families and one or more pathways may include, but are not limited to: dopamine receptor D2 (DRD2), dopamine transporter (SLC6A3 or DAT1), catechol-O-methyltransferase (COMT), and/or monoamine oxidase A (MAOA). Potential treatment for dopamine hypoexpression genes based on the biomarkers examined may include transcranial magnetic stimulation, stimulants, Buproprion, Seligiline, and/or COMT inhibitors. Over expression of this axis, for example in individuals with the COMT Met158Met genotype, leads to a different and unique cluster of symptoms characterized by dopamine over expression, owing to reduced dopamine degradation. These symptoms may include addiction proclivity, dopamine induced mania and the like. Potential treatment for dopamine hyperexpression based on the biomarkers examined may include antipsychotics or S-adenosyl methionine, a COMT agonist which may potentially lower dopamine by enhancing its degradation.

Cognition, memory, excitatory neurotransmission, and long-term potentiation are all related to the strength of synaptic pathways. This axis may include the functional brain circuits in the hippocampus as well as the neurotransmitter systems such as the glutamate neurotransmitter pathway, calcium channels, sodium channels, and the like. Dimensionally, individuals with dysfunction in this axis, herein referred to as LTP-LTD, may exhibit irritability, high recurrence rates, lower thresholds for exacerbations, cyclical mood or cognitive disturbances and the like. Examples of gene markers in this axis may include: alpha-1 subunit of a voltage-dependent calcium channel (CACNA1C or CACH2 or CACN2 or CaV1.2), alpha-1 subunit of a voltage-gated sodium channel (SCN1A or Nav1.1 or FEB3), glutamate transporter (SLC1A1), ankyrin 3 (ANK3 or ANKYRIN-G), and/or brain-derived neurotrophic factor (BDNF). This teaching is meant to point out representative examples of genes affecting the various axes referred to herein, but by no means is a comprehensive listing of all of the potential variation in and amongst these gene families or pathways. Potential treatments for genomic variation leading to excessive excitatory neurotransmission in this axis may include: lithium, Lamotrigine, Valproic Acid, Nimodipine and other calcium channel blockers, memantine, magnesium, Vitamin D or any agent which modulates ionic channels in the brain. Furthermore, the choice whether to use a calcium channel based mood stabilizer or sodium channel modulator may be further assisted by an analysis of these variants. Ankyrin, the gene encoded by ANK3, is enriched at the nodes of ranvier and mediates the aggregate activity of sodium channels in these axonal pathways. Therefore, variants of ANK3 may selectively respond to sodium channel inhibitors such as lamotrigine, riluzole, and other sodium channel modulators.

Information, and particularly biomarker information, indicating dysfunction in any, or preferably all, of these axes may be helpful. Thus any of the systems (including the methods and reports) described herein may include at least one biomarker indicating dysfunction for a particular group, or multiple biomarkers for each group. A three-axis group may also include information on at least one biomarker for each of these pharmacodynamic areas just described.

In some variations a system, method, or article of manufacture may include a fourth axis related to metabolism. This axis may be a pharmacokinetic axis, relevant to metabolism (including drug metabolism). For example, a metabolism area (axis) may include one or more biomarkers for cytochrome P450 mediated hepatic degradation related to pharmacokinetics, methylation, neuroimmune function, blood brain barrier status, brain lipid signaling and insulin pathways. For example representative gene markers may include, but not limited to: cytochrome P450 (CYP2D6, CYP2C19, and CYP3A4), P-glycoprotein (ABCB1), serotonin receptor 2C (HTR2C or 5-HT2C), methylenetetrahydrofolate reductase (MTHFR), melanocortin 4 receptor (MC4R), and/or insulin-degrading enzyme (IDE). This teaching is meant to point out representative examples of genes affecting the various axes referred to herein, but by no means is a comprehensive listing of all of the potential variation in and amongst these gene families or pathways. For example, the list of gene variants described herein is not intended to be exhaustive, but is meant to include other variants that may be linked to the same genetic locus, or variants carrying similar information about the variants mentioned. Near-equivalent variants may be analyzed together to get the same information those mentioned herein.

Thus, in one variation a method, system or article of manufacture may feature at least one biomarker from each of these axes: the autonomic arousal axis, the emotional valence and reward and executive brain function axis, the LTP/LTD synaptic strength axis. In some variations the method, system or article of manufacture may also include one or more marker from the metabolism axis. The clusters of genetic biomarkers related to each (or all) of these axes can be used both for clinical and research purposes. In use, reports indicating the results of biomarkers from these axes can provide a significant amount of information to alert the clinician to a potential abnormality in a prominent neuroanatomical pathway. The pathways implicated in these axes mediate and form the biological basis of behavior, including assessment of external risk and fear (autonomic arousal axis, axis I), novelty seeking, motivation and evaluation of significance (emotional valence, addiction and reward and executive brain function axis, axis II), and synaptic processes/cortical circuits including memory and long term potentiation (axis III).

The systems devices and methods described herein can also be used to help treat the patient by providing patient-specific therapeutic information. This “theranostic” information may allow tailored treatment of neuropsychiatric disorders. Currently, the majority of agents used to treat neuropsychiatric disorders relate to the modulation of serotonin pathways, norepinephrine pathways, dopamine pathways and glutamate pathways; genetic biomarkers associated with these pathways can therefore be employed for treatment decision processes. It may be of particular use to include pharmacodynamic biomarkers along with pharmacokinetic biomarkers. In addition to specific genes related to the metabolism of drugs, the identification of genomic variants related to insulin or lipid metabolism and the like, may lead to the employment of novel therapeutic interventions which are not typically classified as being directly psychotropic. These may include, for example, the use of peroxisome proliferator-activated receptors (PPARs) agonists in bipolar disease, methylfolate for depression associated with variants in MTHFR-related genes, and the like, based on the results of the biomarker assay as interpreted by the systems and methods described herein.

Also included herein are compositions and for the identification and treatment of subjects such that a theranostic approach can be taken to determine the effectiveness of a therapeutic intervention (such as a pharmaceutical or non-pharmaceutical intervention). The methods and reports described herein may allow a reduction in the risk of developing adverse outcomes and may enhance the effectiveness of the intervention. Thus, in addition to diagnosing or confirming the predisposition for a neuropsychiatric illness, the methods and articles of manufacture described also provide a means of optimizing the treatment of a patient by guiding clinicians in choosing appropriate treatments for their patients in a genotypical (or other biomarker) specific fashion. For example, provided herein is a theranostic approach to treating and preventing neuropsychiatric disorders by integrating patient-specific diagnostics and therapeutics to improve treatment of a patient.

In some variations, the dimensional assay may include at least one marker from each of the three axes described (and in some instances, a fourth from metabolic axis). These markers do not, by themselves, indicate a particular diagnosis for a neuropsychiatric disorder (e.g., they are not traditional categorical, or categorical, or etic biomarkers associated with pathway disorders). These areas provide dimensional and phenomenological data about inherited predispositions and vulnerability to pathological states, and thus may provide clinical information useful to treat a variety of neuropsychiatric disorders.

As mentioned above, in some variations the methods, kits and reports described herein provide an integrated analysis of a set of biomarkers, such as genetic markers, epigenetic markers, transcription markers, protein markers, metabolism markers and/or functional brain imaging. The set of biomarkers may be specifically selected to optimize the therapeutic information provided, as described in greater detail herein. The application and incorporation of such a methodology will enhance diagnostic certainty where analysis of any of these markers separately and in isolation provides only limited insight.

One variation of a method of presenting patient-specific and dimensionally based information relevant to the treatment of a neuropsychiatric disorder includes a process for biomarker detection. Any appropriate method for biomarker detection may be used (e.g., gene detection such as SNP, CNV, indel, STR, VNTR, CpG, allelic methylation, etc. . . . ). Biomarker detection may be determined on one or more appropriate platforms. Various platforms may include, but are not limited to Affymetrix, Taqman, Sequenom or Illumina and the like. These platforms can be array-based, PCR-based, or any other single-base detection modalities or sequencing modalities. Development of these platforms can be further validated by robust multi-chip analysis algorithms and subjected to ontological analysis by a variety of different bioinformatics tools. Following detection, a report may be generated, including a description of the physiological significance of the results of the biomarker tests, and additional interpretive information.

The interpretive information provided may include a score or weighting index indicating a confidence level for the interpretive information. This score or index may indicate the number of studies supporting the interpretive comment, the size of the studies supporting the interpretive comment and/or the existence of any disputing or contradictory studies. Some variations, references or links to references may also be provided. Both the testing and the report may be configured to extract patient information most relevant to treatment.

In general, the interpretive analysis may indicate or describe a potential association with a disorder and/or dysfunction of brain activity. For example, if the biomarker for brain dysfunction indicates a genetic variant that is associated with higher rates of disorder or dysfunction (e.g., PTSD, stress disorders, anxiety disorders, etc.), metabolic disorder, etc., as described herein (including in the examples) for each of the various biomarkers indicated.

Several genome-wide association studies (GWAS) have suggested that the combination of several genes, analyzed in a specific cluster, may account for various psychiatric and neurological disorder phenotypes. The observations of such studies are likely incomplete, because they fail to take into account altered protein expression and epigenetic factors. For example, the serum proteome has high complexity with thousands of non-redundant proteins due to multiple post-translational modifications.

Thus, an additional step in the methods and systems for examining and reporting on clinically relevant biomarkers in neuropsychiatry may include an examination of RNA expression. RNA expression analysis may further refine diagnostic specificity as it relates to the actual encoding of DNA in regions of particular interest. Suitable modalities to include are expressed sequence tag analysis and the like.

Another step may include an analysis of the actual protein expression of an altered gene through proteomics. Therefore, one or more proteomic based technologies may be incorporated into the integrative platform described herein. For example, subtractive proteomics, which compares two or more proteomes to identify proteins that are specifically enriched or depleted, may be used as one peptide substrate mapping strategy. Isotope affinity tags are another suitable method of protein detection which may be used.

In some variations DNA methylation analysis may be incorporated into the systems, methods and reports described herein. The methylation status of CpG islands, histones, or allele specific methylation, may correlate with the activity of transcribed genes, which are generally unmethylated. Technologies to assess DNA methylation, including bisulfite reactions are typically hampered by variability, but may benefit from the combined and tiered approach described herein. PCR-based assays and other improvements to the state of the art may also be incorporated into the method, systems, kits and reports described below.

The human genome can be methylated in regions called CpG islands, which control gene transcription through the methylation of methyl-CpG binding domains. When methylated, gene inactivation occurs due to chromatin condensation. In some variations, this epigenetic indicator (e.g., methylation of one or more region of DNA) may be detected and interpreted. Methylation detection methods may determine methylation patterns in a particular genomic locus. Also, specific alleles, some of which are alleles that are part of a SNP, can be methylated. Histones can also be methylated which can impact how a gene is expressed. Various methods can be employed to detect methylation of these genes and their surroundings which is well known to those skilled in the art. For example, Bisulfite methylation tests, methyl-sensitive restriction enzymes, or oligonucleotide reiteration test may be used.

Because CpG motifs are potentially modifiable by environmental factors, they provide a plausible biomarker by which medical interventions may have effects on gene expression. Gene-specific methylation patterns may offer potential molecular signatures of drug responsiveness in psychiatric disorders and may serve as a viable approach for revealing epigenetic processes in a patient's physiology such as medication noncompliance, substance use, hormonal fluctuations, and differences in metabolism.

A number of methods are available for the analysis of CpG methylation levels. Levels of 5-methyl-cytosine (5-mC) can be quantified by enzymatic hydrolysis of the DNA, chromatographic separation of the nucleosides, and analysis by HPLC or MS. The Luminometric Methylation Assay (LUMA) targets all CCGG sequences in the genome by using methylation-sensitive restriction enzymes to discriminate methylated and unmethylated DNA followed by pyrosequencing. Repetitive element sequences, such as Alu, LINE-1 and Sat2, have also been analyzed in a number of studies, as a surrogate for genome-wide methylation levels; such analysis requires bisulfite conversion of cytosine to uracil followed by PCR. In the pyrosequencing analysis of repetitive elements, the PCR step amplifies a given region using primers outside the target sequence containing CpG sites so that both methylated and unmethylated sequences are amplified. This is followed by pyrosequencing of the region of interest. For methylation specific assays such as MethyLight, a Taqman quantitative real-time PCR assay is performed, using primers and probes that are methylation specific and require all CpG sites in the region to be methylated for amplification to occur.

The “methylation density” model suggests that the proportion of methylated cytosines across a region, rather than at any specific position, controls chromatin conformation and thus the transcriptional potential of a given gene. Consistency of DNA methylation patterns is surprisingly strong across many somatic tissues, including brain and lymphocytes. For example, in an examination that included lymphocytes, inter-tissue correlation of 0.95, and suggesting substantial validity for peripheral measurement of DNA methylation as a surrogate for brain methylation status.

Examples of methylation analysis as a “post intervention” biomarker are provided below. These include analysis of the methylation status of the serotonin transporter, COMT, MTHFR and other clusters of functional genes represented in our Axis model. The methylation level of the 5-HTT promoter region can cause effects of 5-HTTLPR on 5-HTT mRNA production. However, this effect is modest and thus, other cis- and trans-acting elements have been proposed to be involved in the regulation of the 5-HTT gene expression, such as the CpG island. Methylation of the promoter region of 5HTT decreases gene expression leading those individuals with long alleles to look more like those with short alleles and carriers of this allele might be particularly likely to show behavioral effects in response to down-regulation resulting from promoter methylation. The weighted average of loci for CpG residues from CpG1 (bp 25586514) to CpG71 (bp 25587180) can be used as index for methylation density in this region. MTHFR T/T genotypes for rs1801133 or hypomethylation of the CpG islands of the MTHFR gene may participate in the control of intracellular Ca(2+) by altering expression in inositol 1,4,5-triphosphate receptor, and S100 calcium binding protein, suggesting that hypomethylation of MTHFR may involve disruption of intracellular calcium. The increased influx of calcium in neurons has been associated as a pathophysiological event in bipolar disease and in particular in individuals with genetic variants of the calcium channel. COMT promoter CpG islands have been detected to be hypomethylated in DNA derived from the saliva in SCZ and BD compared to the control subjects and the observation that S-adenosyl methionine is effective in ameliorating aggressive symptomatology in schizophrenic patients with low catechol-o-methyltransferase (COMT Met158Met variants) supports this notion. CpG-island microarrays have found psychosis-associated DNA-methylation differences in numerous loci, including two hypomethylated glutamate-receptor genes—one near WDR18, located ˜10 kb upstream of the NMDA-receptor-subunit gene NR3B and another in the promoter of the AMPA-receptor-subunit gene GRIA2. Other examples of methylation detection of specific CpG promoter regions associated with the amygdala-HPA axis, the cortical-subcortical dopamine axis, and the ion channel based LTP-LTD channel are also anticipated. Analysis of these critical regions to determine methylation or demethylation of CpG binding domains can be used to determine if a therapeutic intervention has led to a desired effect on gene activation or inactivation.

Methylation detection may be a useful tool as a neuropsychiatric biomarker in the systems, reports and methods described herein for: (1) predicting drug response by measuring gene inactivation in responders versus non-responders in a region of interest; and (2) analyzing markers in disease detection. For example, methylation detection has proven helpful for treatment of lupus patients by observing hypomethylation of DNA in the circulation of these patients. Similarly, the methylation status of GSTP1 is being explored as a marker for prostate or colorectal cancer. Methylation is also being explored as an indicator of drug response. For example, in breast cancer, low methylation of PITX2 in lymph nodes may predict recurrence after Tamoxifen treatment, and in glioblastoma, MCMT methylation may predict response to alkylating agents. In psychiatry and neurology, examples of methylation of particular genes and pathways may also be important, including analysis of BDNF, serotonin transporters and the like. As mentioned above, the combination of an epigenetic indicator, such as methylation, in conjunction with genetic markers in the locus identified herein and/or protein expression makers may prove substantially more powerful and reliable. Thus, an integrative biomarker assay could include specific analysis of gene methylation patterns in critical brain pathways such as those described herein.

In some variation it may be beneficial to examine protein levels and/or expression for one or more biomarkers. For instance, in some the methods and/or articles of manufacture may be configured to examine one or more proteins associated with the inflammatory pathway. Inflammation may be particularly relevant when examining the second axis, which concerns limbic effects (emotional valence, attention, etc.), as inflammation may indicate extensive activation of limbic structures. Biomarkers indicating inflammation or disruption of the blood-brain barrier that may affect the limbic axis may include anti-NMDAr antibodies, S100Beta, MMP-9, antinuclear antibodies (ANA), etc. These biomarkers maybe particularly helpful in treating psychotic states, particularly in the acute phase, when the markers may be up-regulated. In any of the variations described herein a biomarker may include a protein that is examined as described. For example, brain imaging modalities which can provide relevant clinical data regarding neuropsychiatric symptomatology across the axis described above may include: fMRI, DTI, and MRS (magnetic resonance spectroscopy). For instance, diffusion tensor imaging demonstrated decreased white matter integrity, indicated by lower fractional anisotropy and longitudinal diffusivity, in the ANK3 rs10994336 risk genotype in the anterior limb of the internal capsule. Further examples include association of the val(158) allele with lower blood oxygen level-dependent (“BOLD”) response in ventromedial and dorsomedial prefrontal cortex compared to val(158) non-carriers, whereas met(158) homozygotes showed lower BOLD response in the posterior cingulate and precuneus compared to val(158) carriers compatible with a hypothesis on the role of COMT val(158) met genotype in tonic and phasic dopamine levels in brain. Further examples are reported herein.

In some variations, the biomarkers may be examined for a particular patient over time. For example, biomarkers in any or all of the axes examined may be analyzed before, during or after a treatment. Thus, the methods and articles of manufacture may be used to monitor treatment and/or progression of a neuropsychiatric disorder.

In general, any of the genes described herein may be used as biomarkers by testing for polymorphisms, mutations, insertions, deletions, translocations, methylation, histone methylation and/or deacetylation, etc. The proteins expressed by any of these genes may also be tested for expression level, localization, folding (or miss-folding), and the like.

The methylation status, including hypo- and hyper-methylation of certain genes may be a marker of neuropsychiatric disorder. In general, epigenetic modulations may play an important role in fine-tuning of gene expression in response to environmental factors. For instance, using quantitative methylation specific PCR, MB-COMT promoter has been seen to be hypo-methylated in DNA derived from the saliva in schizophrenia compared to control subjects, suggesting that DNA methylation analysis of MB-COMT promoter in saliva can potentially be used as an epigenetic biomarker for disease state. Further, the CpG at T102C of the HTR2A polymorphic site and neighboring CpGs were approximately 70% methylated both in the patients and controls. qMSP analysis revealed that the cytosine of the T102C polymorphic site was significantly hypo-methylated in SCZ compared to the controls. Thus, for example, Cytosine methylation of HTR2A at T102C polymorphic site in DNA derived from the saliva can potentially be used as a diagnostic, prognostic, and/or therapeutic biomarker in psychiatric conditions associated with psychotic symptoms.

As another example, the methylation status of retinoic acid-related orphan receptor alpha (RORA) has been implicated in Autism. Methylation of RORA was confirmed by bisulfite sequencing and methylation-specific PCR; this data has revealed decreased expression of RORA proteins in the autistic brain.

Methylation may also be used as a biomarker for heightened stress, intractable anxiety and other clusters of symptoms related to the amygdala-HPA axis. For example, SLC6A4 methylation levels appear to modify the effect of the number of traumatic events on PTSD after controlling for SLC6A4 genotype. Persons with more traumatic events were at increased risk for PTSD, but only at lower methylation levels. At higher methylation levels, individuals with more traumatic events were protected from this disorder. Depressive symptoms were more common among those with elevated buccal cell 5HTT methylation who carried 5-HTTLPR short-allele. Thus hypomethylation of SLC6A4 may be used as a marker of depression and/or PTSD.

Protein expression may also be used as a biomarker. The examination of protein expression, including proteomics, may use an analytic method such as mass spectrometry, nanostring, and the like. Protein expression may also be examined by immunological methods (e.g., immunocytochemical detection). An abnormal protein may correspond to an abnormal biological state, whereas a gene abnormality is more trait dependent. Proteins that are found to be more prevalent in diseased patient samples compared to normal patient samples may be an important potential disease biomarker for disorders like dementia, schizophrenia, autism, bipolar, anxiety, depression and the like. However, a search for any particular biomarkers in disease-free or asymptomatic individuals is neither cost effective nor efficient. Therefore, it may be significantly more effective to combine an assessment of genetic risk and/or epigenetic risk with a proteomic analysis.

It should be noted that there currently exist commercial assays which are used for psychiatric diagnosis. A clear distinction between the systems, reports and methods described herein and these other tests includes the difference between analyzing pharmacodynamic (PD) genes and pharmacokinetic (PK) genes. In the latter example, PK genes provide information related to drug metabolism but do not provide any insight into trait dependent and specific neurochemical factors related to neuropsychiatric conditions. These trait-dependent factors, which are components of the current disclosure, include assessment of stress resilience, risk of impairments in reward mediated behaviors, risk of psychiatric decompensation or cyclical mood disturbances, subendophenotypes of depression, and the like. The systems described herein may examine biomarkers indicative of pharmacodynamic (PD) traits. These biomarkers test for the activity or interactions of one or more members of a biological pathway, including those pathways involved in neurotransmission. For example, the methods described herein may examine genes related to neurochemical imbalances. Such tests may be broadly applied to the genes involved in at least the following pathways: Serotonin, dopamine, norepinephrine, glutamate and the hypothalamic pituitary adrenal axis. Additional gene analysis also relates to calcium channels, sodium channels, potassium channels which are also relevant to neuropsychiatric disorders and response to particular interventions. Other genes of importance relate to metabolism. These genes include brain glucose utilization, methylation, inflammation and the like.

Specific genes within these areas are described in the paragraphs herein but are not limited to this disclosure. Thus, while the present invention describes polymorphisms in a specific serotonin gene, it is recognized that other polymorphisms in the serotonin pathway are contemplated as within the scope of this disclosure. Similarly, biomarkers in the glutamate pathway, dopamine pathway, norepinephrine pathway, and HPA axis may be examined as well. Gene detection such as SNPs, CNVs, indels, STRs, VNTRs and the like on various platforms known to those skilled in the art, such as the Affymetrix, Taqman, Sequenom or Illumina, can be utilized.

There are many variations of target nucleic acid amplification, including, for example, polymerase chain reaction (PCR), which has been disclosed in numerous publications. The most commonly used target amplification method is the polymerase chain reaction (PCR), which consists of repeated cycles of DNA polymerase-generated primer extension reactions. Each reaction cycle includes heat denaturation of the target nucleic acid; hybridization to the target nucleic acid of two oligonucleotide primers, which bracket the target sequence on opposite strands of the target that is to be amplified; and extension of the oligonucleotide primers by a nucleotide polymerase to produce multiple, double-stranded copies of the target sequence. The discovery of thermostable nucleic acid modifying enzymes has contributed to rapid advances in nucleic acid amplification technology.

The exemplary systems, screens and methods described herein may include assays for determining genetic indicators (including genetic polymorphisms), epigenetic markers (such as methylation status), and protein expression. Such assays may include known tests, assays or methods, which may be integrated or combined in known or novel ways. For example, diagnostic kits using “gene chip” technology may be used to determine genetic and/or epigenetic information about particular genes of interest, and may be integrated with protein indicators including immunoassays or the like.

For example, in some variations a neuropsychiatric-specific oriented kit may be provided. This kit (which may include an array) may be built to provide a practical and clinically relevant tool in practice in neuropsychiatry because it is able to translate GWAS-level research findings into a clinically practical framework. In this fashion, the benefit of focusing on a narrow and pre-selected group of SNPs, CNVs, repeats or indels relates to the application and context of the results in an integrative clinical setting.

As a specific example of the methods of diagnosing and/or treating a neuropsychiatric diagnosis described above, the inventor has applied one variation to identify psychiatric disorders based upon epistasis between 2 or more genes. For example, the inventor has discovered that individuals with a COMT Val/Val polymorphism in epistasis with the MTHFR T/T variant may display a phenotype characterized by a subcortical-type of mood disorder. These individuals commonly are abulic, dysthymic, and allergic. This phenotype may be expressed secondary to reduced prefrontal dopamine as a consequence of these genes being in epistasis, resulting in excess dopamine degradation. Thus, a system, report or method may examine the combination of COMT and MTHFR and/or dopamine neurotransmitter pathway genes. One or more genetic markers, epigenetic markers and/or protein expression may be examined to determine if a patient has or is at risk for the correlated abulic, dysthymic, and anergic phenotype.

In another example, the combination of serotonin short alleles and CACNA1C variants has also been linked by the inventor to a particular phenotype which may be specifically amenable to treatment, either to enhance treatment or to select between available treatments that would otherwise be seemly equivalent based only on the phenotype presented to the physician. For example, SSRI-induced mania may be higher in these patients.

For example, described herein are methods of presenting patient-specific dimensional PD information relevant to the treatment of a neuropsychiatric disorder. These methods may be used to improve psychiatric diagnosis, including depression, bipolar, schizophrenia, dementia, PTSD, anxiety and the like. In general, any of the methods, systems, and articles of manufacture described herein may use a specified cluster of biomarkers. As described herein, these clusters of biomarkers may include, for example, a representative set of biomarkers having particular relevance across a cross-section of neurotransmitter pathways, neurofunctional pathways, and/or neuroanatomical pathways. These biomarkers may be derived from the compacting and compression method described herein. For example, the biomarkers may include one or more biomarkers from each of the patient's autonomic arousal system, the patient's emotional valence, attention, reward and executive brain functions, and the patient's cognition and memory systems. Biomarkers examined may include SNPs, CNVs, indels, STRs, VNTRs, methylation, protein expression, functional brain MRI and the like. The selection of biomarkers, particularly those described in greater detail below, may indicate that status or functionality of neurotransmitter pathways, the patient's neuroimmune system and/or neuroendocrine system. The methods described herein may provide an integrative framework applied to these biomarkers in which component elements are interpreted in a holistic neural net framework, rather than reductionist fashion.

Further, any of the methods described herein may incorporate specific brain imaging modalities, including magnetic resonance spectroscopy (functional, tensor, etc.) and the like. The incorporation of these biomarkers and their interpretation in clinical practice is also described. In particular, the devices, systems and methods described herein allow interpretation of key subsets of biomarkers which address the translation of research findings into clinically meaningful data. For example, the systems, methods and reports described herein provide both raw biomarker test results for a specific and meaningful group of biomarkers, as well as interpretive data including clinical and research findings specific to the patient's biomarker test results. In some variations this interpretive data is ranked, weighted or indexed to provide a confidence level to the physician or medical professional. Thus, the methods, devices and systems described herein may provide clinical support which includes specific educational material for patients and/or clinicians.

Further, the methods, devices and systems described herein may provide analytical methods to enhance the signal to noise ratio related to the use of biomarkers in psychiatry.

In some variations, the methods of presenting patient-specific PD information relevant to the treatment of a neuropsychiatric disorder include the steps of: providing a patient identifier; presenting a description of a biomarker test result specific to the patient; presenting an interpretive analysis of the neurophysiological significance of the biomarker test result for the patient, wherein the interpretive analysis comprises pharmacodynamics information; and presenting a weighted index of confidence level for the interpretive analysis.

These methods may be used in order to improve treatment, and in some variations, may also be used to help identify and/or diagnose patients. In some variations, the methods may be used to help delineate specific treatment interventions based upon the results of the biomarkers.

The step of providing a patient identifier may include generating a report including any patient identifying mark, code, name, symbol, or the like. For example, the patient identifier may include a patient number or patient name. The patient name may be kept confidential in some variations. In variations in which the method includes providing a copy of the results, the results copy may include a written patient identifier as part of the copy of the results.

The step of presenting a description of a biomarker test result specific to the patient may include a listing or output of the raw result of the biomarker test and/or an amended form of the results. For example, when SNPs are used, the presence or absence of the derived allele on each chromosome may be provided. In some variations, the raw biomarker test result is not provided, but only a summary of the result is included (e.g., “the patient tested positive for . . . ” a particular biomarker). In some variations, the test results may indicate a polymorphism, deletion, duplication, insertion, methylation level, methylated allele, expression level, expression localization, activity, or metabolites of one or more gene, protein, or neurotransmitter.

In general, any of the steps of presenting information (e.g., presenting a description, presenting an interpretive analysis, presenting a weighted index, etc.) may include generating a report including the presented information, or including the presented information on a single report. As discussed herein, the report may be a single page or multiple pages, both in written or digital formats.

The step of presenting an interpretive analysis of the neurophysiological significance of the biomarker test result for the patient, wherein the interpretive analysis comprises PD information, may include providing any appropriate type of interpretive analysis and comments. Appropriate interpretive analysis typically includes a description of the physiological significance of the biomarker test result. For example, the interpretive analysis may indicate associations with neuropsychological disorders, drug response, patient behaviors, treatment outcomes, or the like in patients with the same biomarker test results. The interpretive analysis may also include a description of the gene and/or protein, and/or biological pathway associated with the particular biomarker. In some variations the interpretive analysis may also include association studies, such as gene response association studies, describing or summarizing research and/or clinical studies on the biomarker and any associations based on the presence and/or absence of the biomarker.

In some variations the interpretive analysis may also include a visual representation of a region of the patient's brain affected by the underlying biomarker (e.g., the gene and/or protein being tested by the biomarker test). The visual representation may be generic (e.g., not taken from the actual patient's brain). Multiple visual representations (including alternative views, color views, animations, etc.) may be provided. The interpretive analysis may also include possible drug responses.

The results may be provided in hard copy (e.g., written form) or they may be electronic, including delivered as a web page, PDF, or other “virtual” document.

The step of presenting a weighted index of confidence level for the interpretive analysis may include indicating for all or some of the interpretive analysis an approximation of the confidence level for that particular portion of the interpretive analysis. For example, an index may include a “score” based on the reproducibility (or lack of reproducibility), the number of patients/subject's examined in the academic or clinical literature or references, the length of time studied, or the like. In general, these confidence level scores may be summarized in the report in a key, or they may be self-qualifying (e.g., the index may indicate “high,” “medium” or “low” confidence values). In some variations the weighted index of confidence level may include alphanumerically indexing all or a portion of the interpretive analysis with a score indicating the type and/or number of studies supporting the interpretive analysis.

Any neuropsychiatric disorder may be addressed by the methods, devices and articles of manufacture described herein, particularly for treatment resistance. For example, the neuropsychiatric disorder examined may be selected from the group including: treatment resistant depression, bipolar depression, anxiety disorders, PTSD, schizophrenia, dementia, autism, and ADHD. In some variations the patient may not be diagnosed with a particular neuropsychiatric disorder; in some variations the methods, systems and reports described herein may be used as an aid in treating the patient.

Although the general method of presenting patient-specific PD information relevant to the treatment of a neuropsychiatric disorder includes only a single biomarker test result, it is of particular interest to examine and present patient-specific information about a set of biomarkers. In particular a set of biomarkers that include one or more markers from a subset of “axes” such as the patient's autonomic arousal system, the patient's emotional valence, attention, reward and executive brain functions, and the patient's cognition and memory systems. Each of these axes describes PD biomarkers; in some variations it may also be helpful to include one or more markers of PK biomarkers. Examples of specific markers are provided herein. In particular, depression (and treatment-resistant depression especially) may include one or more markers from each of patient's autonomic arousal system, the patient's emotional valence, attention, reward and executive brain functions, and the patient's cognition and memory systems. When examining other neuropsychiatric disorders, only one or two of these axes may be used, or entirely other axes may be chosen.

As mentioned, in some variations the biomarker provides information about the autonomic arousal system of the patient's brain. For example, the biomarker may be a marker of a gene, or a protein encoded or modulated by gene selected from the group consisting of: SERT, SLC6A4 (SERT), HTR1A, ACE, NPY, FKBP5, and other genes associated with heightened amygdala function. In some variations a biomarker provides information about the emotional valence, attention, reward and executive brain functions of the patient. For example, the biomarker may be a marker of a gene, or a protein encoded or modulated by gene selected from the group consisting of: COMT, sigma receptors, SNAP25, MAOA, SLC6A3, and DRD2. In some variations, the biomarker provides information about the strength of synaptic pathways (LTP). For example, the biomarker may be a marker of a gene, or a protein encoded or modulated by gene selected from the group consisting of: CACNA1C, SLC1A1, ANK3, and BDNF.

Also described are methods of presenting patient-specific PD information relevant to the treatment of a neuropsychiatric disorder including the steps of: presenting a description of a biomarker test result specific to a patient for at least one biomarker related to each of: the patient's autonomic arousal system, the patient's emotional valence, attention, reward and executive brain functions, and the patient's cognition and memory; and presenting an interpretive analysis of the neurophysiological significance of each biomarker test result for the patient, wherein the interpretive analysis comprises PD information. All of the variations and additional steps described above may also be applied to these methods.

Also described herein are methods of presenting patient-specific pharmacodynamics information relevant to the treatment of a neuropsychiatric disorder, the method comprising: providing a patient identifier; presenting a description of a plurality of biomarker test results specific to the patient; presenting an interpretive analysis of the neurophysiological significance of each of the biomarker test result for the patient, wherein the interpretive analysis comprises pharmacodynamics information; and presenting a visual representation of a brain region affected by each biomarker.

Articles of manufacture for assisting in the treatment of neuropsychiatric disorders are also described herein. For example, described herein are articles of manufacture comprising an interpretive neuropsychiatric report of patient-specific pharmacodynamics information relevant to the treatment of a neuropsychiatric disorder, the article of manufacture comprising: a report including a patient identifier; a description of a biomarker test result specific to the patient; an interpretive analysis of the neurophysiological significance of the biomarker test result for the patient, wherein the interpretive analysis comprises pharmacodynamics information; and a weighted index of confidence level for the interpretive analysis. The report is generally written, and the tangible medium of the report may be hardcopy (e.g., paper) or electronic (e.g., a digital file describing the written results). Thus, in any of the articles of manufacture described, the patient identifier and descriptions of the biomarker test results and interpretive analysis may be non-transiently formed on the report. The report may also be stored in any appropriate electronic medium (e.g., digital medium).

In some variations, the article of manufacture includes a plurality of descriptions of biomarker test results specific to the patient for a plurality of biomarkers, and may also include interpretive analyses of the neurophysiological significance of each of the biomarker test results for the patient.

As mentioned above, the interpretive analysis may further comprise a description of the physiological significance of the biomarker test result for the patient, a description of published studies describing similar biomarker test results, an indicator of possible drug responses, and/or a visual representation of a brain region affected by the biomarker.

In addition to the pharmacodynamics biomarker(s), the article of manufacture may also include a description of a biomarker test results for a pharmacokinetic biomarker.

The weighted index of confidence level may include an alphanumerical index of all or a portion of the interpretive analysis with a score indicating the type and/or number of studies supporting the interpretive analysis. The article of manufacture may also include a list of references specific to the patient's biomarker test result (the references may be part of the interpretive analysis).

As mentioned above, the biomarker test result may indicate a polymorphism, deletion, repetition, insertion, methylation, expression level, expression localization, activity, or metabolites of one or more gene, protein, or neurotransmitter. For example, an article of manufacture may include a test result and interpretive comments for a biomarker related to the autonomic arousal system of the patient's brain, such as a gene, or a protein encoded or modulated by gene selected from the group consisting of: SLC6A4 (SERT), HTR1A, ACE, NPY, and FKBP5. An article of manufacture may include a test result and interpretive comments for a biomarker related to the emotional valence, attention, reward and executive brain functions of the patient, such as a gene, or a protein encoded or modulated by gene selected from the group consisting of: COMT, MAOA, SNAP25, SLC6A3, and DRD2. An article of manufacture may include a test result and interpretive comments for a biomarker related to the patient's cognition and memory, such as a gene, or a protein encoded or modulated by gene selected from the group consisting of: CACNA1C, SLC1A1, ANK3, and BDNF.

In some variations of the articles of manufacture described herein, the article of manufacture may include an interpretive neuropsychiatric report of patient-specific pharmacodynamics information relevant to the treatment of depression, the article of manufacture comprising: a report including a description of a biomarker test result specific to a patient for at least one biomarker related to each of: the patient's autonomic arousal system, the patient's emotional valence, attention, reward and executive brain functions, and the LTP-LTD axis; and an interpretive analysis of the neurophysiological significance of each biomarker test result for the patient, wherein the interpretive analysis comprises pharmacodynamics information. The article of manufacture may also include a weighted index of confidence level for all or part of each interpretive analysis.

In some variations of the articles of manufacture described herein, the articles include an interpretive neuropsychiatric report of patient-specific pharmacodynamics information relevant to the treatment of a neuropsychiatric disorder, the article of manufacture comprising a report including: a patient identifier; a description of a plurality of biomarker test results specific to the patient; an interpretive analysis of the neurophysiological significance of each of the biomarker test result for the patient, wherein the interpretive analysis comprises pharmacodynamics information; and a visual representation of a brain region affected by each biomarker.

Also described herein are methods of diagnosing a neuropsychiatric disorder based on patient-specific dimensional information. For example, the methods may include the steps of: sampling a patient; testing the sample for at least one biomarker related to each of: the patient's autonomic arousal system, the patient's emotional valence, attention, reward and executive brain functions, and the patient's LTP-LTD axis; providing a report including the results of the biomarker test, an interpretive analysis of the neurophysiological significance of each biomarker test result, and a weighted index of confidence level for the interpretive analysis.

Systems for performing the methods described herein are also included, as are systems for generating the articles of manufacture (e.g., reports) mentioned above. For example, a system for generating a patient-specific pharmacodynamics report relevant to the treatment of a neuropsychiatric disorder may include: an input module configured to receive at least one biomarker test result specific to a patient for each of: the patient's autonomic arousal system, the patient's emotional valence, attention, reward and executive brain functions, and the patient's cognition and memory; an analysis module coupled to the input module and configured to generate an interpretive report from the plurality of biomarker test results, wherein the analysis module generates interpretive comment for each biomarker based on the test result.

Also described herein are systems for diagnosing or guiding a therapeutic treatment of a neuropsychiatric disorder comprising: an assay for determining the status of at least one biomarker related to each of: the patient's autonomic arousal system, the patient's emotional valence, attention, reward and executive brain functions, and the patient's LTP-LTD axis; and a report including the status of the biomarkers determined, an interpretive analysis of the neurophysiological significance of each biomarker's status, and a weighted index of confidence level for the interpretive analysis.

Also described herein are methods for simplifying and presenting patient-specific treatment information for the treatment of a psychiatric disorder, as well as customized reports presenting information to guide treatment of psychiatric disorders. In particular, described herein are reports and methods for presenting information on the treatment of treatment resistant psychiatric disorders based on patient-specific information.

In general, the methods of presenting information and the presentations (e.g., reports) described herein include the presentation of patient-specific data from a core set of genetic loci which the inventors have found to be critical to guiding the treatment of resistant forms of psychiatric disorders. Thus, the presentation provides epistatic information related to the core areas, axes, or loci discussed above. The axes (loci) may be referred to functionally (e.g., cognition and memory, etc.), neuroanatomically (e.g., hippocampal, limbic, etc.) or based on their principle neurotransmitter pathway (dopaminergic, glutamatergic, etc.).

For example, described herein is a method of presenting patient-specific treatment information for treatment resistant depression may include: presenting the patient-specific information for each of the core genetic loci in an epistatic group, and presenting interpretive comments for each the results. As just mentioned, the genetic loci forming a core epistatic group typically relate to genes/proteins having a functional relationship for a particular neurotransmitter pathway, and/or neuroanatomical location, and/or neurological function.

The methods and reports described herein may present the biomarker results for a patient (e.g., a patient genotype) in a single report including biomarker information from each or the four axes identified (or a subset of them), and also present interpretive comments based on the results. The interpretive comments may describe a likely drug response based on the outcome of the biomarker results. For example, a report may provide the genotypes for biomarkers of a particular epistatic locus, and may describe putative or definite links between the results of one or more biomarker and an expected clinical significance. The interpretive comments may describe the function of a particular gene generally, and may specifically describe the significance of the genetic result of the biomarker test for that gene (or all relevant outcomes/genotypes).

In some variations, the systems and methods may provide an interpretation of the results of an analysis of the ACE gene. The report may indicate that one or more ARBs (e.g., losartan, telmisartan, etc. or a pharmaceutical composition including one or more ARB) should be used to treat the patient. Thus the use of an angiotensin receptor blocker (ARB) composition may be determined based on one or more genetic markers that may be useful in predicting PTSD symptom reduction. In some variations, patients that are CC homozygotes for rs4311 SNP in the ACE gene may be reported to have a superior response to ARBs on PTSD symptoms compared to T carriers.

Also described herein are articles of manufacture based on the concepts taught herein. One particular article of manufacture contemplated herein is a written or displayed report describing a relevant set of biomarkers, the results of the biomarker tests, and interpretive comments including in some variations genetic information that is patient-specific and relevant to treatment of a neuropsychiatric disorder. In some variations the report includes a section providing the patient's genotype. The report may also include interpretive comments for each of the axes tested. Finally, the report may include a weighting index that provides a confidence level for all or some of the interpretive comments.

In some variation the report is an electronic report. In other variations the report is a written report. The report may be coded to indicate the presence of a genetic polymorphism in each member of the core epistatic group. The report may also include a summary (e.g., a table, chart, etc.) that lists and summarizes the genotype test results; this summary may be on the first page or the top/front of the report.

The interpretive comments may be included for each biomarker examined after a description of the genotype result for that member. In general, interpretive comments may include treatment recommendations, references to scientific literature, and any other statement describing the significance of one or more genotype. Interpretive comments may provide interpretation of the significance of each of the genotypes. Interpretive comments may also provide interpretation of the significance of combinations of genotypes for different biomarkers tested, particularly those within the different axes. Interpretive comments may also provide information on the significance of particular patient phenotypes in combination with specific (including patient-specific) genotypes.

In some variations, the interpretive comments include a visual representation of the effected brain region, including a representational image of the neuroanatomical region affected by a polymorphism identified by a biomarker, for example.

Interpretive comments may be tailored to correspond to the biomarker result for an individual; in some variations, the interpretive comments are generically provided regardless of the biomarker result. In variations in which all of the possible interpretive comments are provided regardless of the biomarker results, interpretive comments that are relevant to the identified biomarker result may be highlighted.

In general, the report may highlight or separate out the biomarker results, particularly when the biomarker indicates the presence of a polymorphism or risk factor having therapeutic consequences. For example, in some variations results indicating polymorphisms may be highlighted. Highlighting may include presenting the text in a different font, color, point, or the like, including (but not limited to) boxing the text, indenting the text, boding the text, italicizing the text, underlining the text, or the like.

In one variation of the methods of presenting patient-specific and dimensionally based information relevant to the treatment of a neuropsychiatric disorder described herein, the method may include the steps of: presenting written biomarker test results specific to a patient for at least one biomarker for dysfunction in each of the following axes: (1) a patient's autonomic arousal system; (2) the patient's emotional valence, attention, reward and executive brain functions; and (3) the patient's long-term potentiation and long-term depression (LTP-LTD) function; and presenting an interpretive analysis of the neurophysiological significance of each biomarker test result for the patient, wherein the interpretive analysis comprises patient-specific information on response to a neurotherapeutic agent based on the biomarker test results.

In some variations, this method is directed specifically to methods of presenting patient-specific and dimensionally based information relevant to the treatment of depression or treatment-resistant depression.

In some variations, the step of presenting the interpretive analysis comprises presenting an association with a neuropsychiatric condition based on the biomarker test results. In general the methods and articles of manufacture described herein are useful for providing relevant and helpful treatment information that is not necessarily linked to a particular or specific diagnosis, and the methods described herein do not necessarily provide a diagnosis or a categorical diagnosis. Thus, there may be overlap or comorbid presentation of symptoms. In some variations, the methods and articles of manufacture may be used in conjunction with a traditional categorical diagnosis. For example, these methods and articles of manufacture may be used to aid in the treatment of patients suspected or identified as having a neuropsychiatric disorders selected from the group including: treatment resistance associated with depressive disorders, bipolar disorder, anxiety disorders, PTSD, schizophrenia, autism, and ADHD.

In any of the methods and articles of manufacture described herein, the step of presenting the interpretive analysis may further comprise presenting a description of a neurophysiological correlation with the biomarker test results. In particular, the method or article of manufacture may include a description (including a picture or visual representation) of a region of the brain affected by particular status of the biomarker. In some variations the methods and articles of manufacture include a visual representation of a brain region relevant to each biomarker (e.g., highlighting the neuroanatomical regions of pathways affected by variations or disruptions in the genes informed by the biomarker). For example, if a biomarker indicates a potential dysfunction in one of the three neuropsychiatric axes identified (e.g., the autonomic arousal axis, the emotional valence, attention, reward and executive brain function axis, and/or the long-term potentiation and long-term depression axis), the interpretive analysis may provide correlated clinical findings relevant to that potential dysfunction. The interpretive analysis may also specifically address treatment regimes, including any relevant therapeutic (e.g., drug, medical foods, etc.) interactions or suggestions. The interpretive analysis may indicate if one or more therapeutics is indicated or contraindicated given the biomarker result(s).

Any of the variations of methods and articles of manufacture may also provide a referral to a call center to receive additional interpretive information. Thus, in some variations, the methods may include a phone number, web address, or other contact information, and/or instructions for contacting a call center to receive additional information on the results of the biomarker testing. The call center may be staffed or automated, and generally provides additional expert advice and information on the biomarker results.

In general the step of presenting may include presenting written biomarker test results specific to a patient for a pharmacokinetic biomarker. The written results may be electronic (“virtual”) or paper (“real”) and may be delivered or accessible to a medical physician. The results may be secured so that they can be accessed only by a physician and/or in some variations the patient.

As mentioned, in some variations it may be helpful to include additional biomarkers outside of the three neuropsychiatric axes mentioned. In particular, it may be helpful to include biomarkers for pharmacokinetic (e.g., drug metabolism) pathways, such as cytochrome P450.

In general, the biomarker test results may indicate polymorphism, deletion, repetition, insertion, methylation level, allele specific methylation, expression level, expression localization, activity, or metabolites of one or more gene, gene family, pathway, transcript, protein, or neurotransmitter.

A biomarker for dysfunction in the patient's autonomic arousal system axis may be a marker of a gene, or a protein encoded or modulated by gene, selected from the group consisting of: SLC6A4 (SERT), ACE, NPY, FKBP5, and HTR1A. Similarly, a biomarker for dysfunction in the patient's emotional valence, attention, reward and executive brain functions axis may be a marker of a gene, or a protein encoded or modulated by gene, selected from the group consisting of: COMT, SLC6A3, and DRD2. A biomarker for dysfunction in the patient's long-term potentiation and long-term depression (LTP-LTD) function axis may be a marker of a gene, or a protein encoded or modulated by gene, selected from the group consisting of: CACNA1C, SLC1A1, ANK3, BDNF, and APOE.

Presentation of a biomarker for disruption of each of the autonomic arousal axis, the emotional valence, attention, reward and executive brain function axis, and the long-term potentiation and long-term depression axis provides a surprisingly compete picture for treating neuropsychiatric disorders. It may be important to include all three of these axes in the methods and articles of manufacture described herein because the combination of these three axes allows an accurate approximation of the majority of therapeutic interactions for neuropsychiatric treatments; although additional axes and/or biomarkers within these axes could be included, including at least one biomarker from each of these three axes allows the methods and articles of manufacture described herein to provide interpretive information relevant to the majority of therapeutic treatments, independent of the categorical classification of the diagnosis or suspected diagnosis.

For example, the step of presenting the interpretive analysis may include predicting the patient's response to a neurotherapeutic agent selected from the group consisting of: ARBs and/or pharmaceutical compositions including one or more ARB (as described in part 2, below), Lithium, norepinephrine modulators, dopamine augmenting agents, monoamine oxidase inhibitors, COMT inhibitors, S-adenosyl methionine, mood stabilizers, calcium channel agents, Racetam agents, Tianeptine or Transcranial magnetic stimulation. Thus the interpretive analysis may specifically describe a patient's likely response/outcome for one or more (or all) of these therapeutic agents based on the results of the biomarkers described.

In some variations the methods and articles of manufacture include a survey or patient response questionnaire to be completed by the patient or the patient's physician addressing patient history and/or behavior. In some variations this information may help inform the analysis of the biomarkers, or it may be used to track the effect of a therapy. For example, a questionnaire may assess mood problems, memory difficulty, anxiety issues and health related issues in psychiatric patients, particularly those with two or more medication failures. Thus, in some variations of the method described herein, the method may also include the step of requesting patient treatment history information.

In one variation a method of presenting patient-specific and dimensionally based information relevant to the treatment of a neuropsychiatric disorder includes the steps of: presenting in a report a description of a biomarker test result specific to a patient for at least one biomarker related to the patient's autonomic arousal system axis, wherein the biomarker is related to a gene or a protein encoded or modulated by a gene selected from the group consisting of: SLC6A4 (SERT), ACE, NPY, FKBP5, and HTR1A (and/or MDR1); presenting in the report a description of a biomarker test result specific to the patient for at least one biomarker related to the patient's emotional valence, attention, reward and executive brain function axis, the biomarker related to a gene or a protein encoded or modulated by a gene selected from the group consisting of: COMT, SLC6A3, and DRD2; presenting in the report a description of a biomarker test result specific to the patient for at least one biomarker related to the patient's long-term potentiation and depression (LTP-LTD) function axis, the biomarker related to a gene or a protein encoded or modulated by a gene selected from the group consisting of: CACNA1C, SLC1A1, ANK3, BDNF, and APOE; presenting in the report an interpretive analysis of the neurophysiological significance of the patient's autonomic arousal system axis biomarker test result, the patient's emotional valence, attention, reward and executive brain function axis test result, and the patient's long-term potentiation and long-term depression function axis, wherein the interpretive analysis comprises patient-specific information on response to a neurotherapeutic agent based on the biomarker test results; providing a visual representation of a brain region relevant to each biomarker; and providing a referral to a call center to receive additional interpretive information.

Also described herein are articles of manufacture comprising an interpretive neuropsychiatric report of patient-specific and dimensional information relevant to the treatment of a neuropsychiatric disorder, the article of manufacture comprising: a written description of a biomarker test result specific to a patient for at least one biomarker for dysfunction in each of the following axes: (1) the patient's limbic based autonomic arousal system; (2) the patient's pre frontal-subcortical emotional valence, attention, reward and executive brain functions; and (3) the patient's synaptic mediated long-term potentiation and long-term depression (LTP-LTD) function; and an interpretive analysis of the neurophysiological significance of each biomarker test results for the patient, wherein the interpretive analysis comprises patient-specific information on response to a neurotherapeutic agent based on the biomarker test results. As mentioned, the interpretive analysis may include a prediction of the patient's response to a neurotherapeutic agent, and particular a neurotherapeutic agent selected from the group consisting of: Lithium, norepinephrine modulators, angiotensin receptor blockers, dopamine augmenting agents, monoamine oxidase inhibitors, COMT inhibitors, S-adenosyl methionine, mood stabilizers, calcium channel agents, Racetam agents, Tianeptine or Transcranial magnetic stimulation.

The article of manufacture may also include a written description of a biomarker test results for a pharmacokinetic biomarker (e.g., cytochrome P450, etc.).

As mentioned, any of the methods and articles of manufacture described herein may include a referral to a call center for receiving additional interpretive information. The article of manufacture may be electronic or printed. In general, the article of manufacture may indicate polymorphism, deletion, repetition, insertion, methylation level, allele specific methylation, expression level, expression localization, activity, or metabolites of one or more gene, gene family, pathway, transcript, protein, or neurotransmitter.

For example, the biomarker for dysfunction in the patient's autonomic arousal system axis may be a marker of a gene, or a protein encoded or modulated by gene, selected from the group consisting of: SERT, SLC6A4 (SERT), 5HT1a, ACE, NPY, FKBP5, and HTR1A.

The biomarker of the article of manufacture for dysfunction in the patient's emotional valence, attention, reward and executive brain functions axis may be a marker of a gene, or a protein encoded or modulated by gene, selected from the group consisting of: COMT, SLC6A3, and DRD2. The biomarker for dysfunction in the patient's long-term potentiation and long-term depression (LTP-LTD) function axis may be a marker of a gene, or a protein encoded or modulated by gene, selected from the group consisting of: CACNA1C, SCN1A, ANK3, and BDNF.

Also described herein are articles of manufacture comprising an interpretive neuropsychiatric report of patient-specific and dimensional information relevant to the treatment of a neuropsychiatric disorder, the article of manufacture comprising: a written description of a biomarker test result specific to the patient for at least one biomarker related to the patient's autonomic arousal system axis, the biomarker related to a gene or a protein encoded or modulated by a gene selected from the group consisting of: SLC6A4 (SERT), ACE, NPY, FKBP5, and HTR1A; a written description of a biomarker test result specific to the patient for at least one biomarker related to the patient's emotional valence, attention, reward and executive brain function axis, the biomarker related to a gene or a protein encoded or modulated by a gene selected from the group consisting of: COMT, SLC6A3, and DRD2; a written description of a biomarker test result specific to the patient for at least one biomarker related to the patient's long-term potentiation and long-term depression (LTP-LTD) function axis, the biomarker related to a gene or a protein encoded or modulated by a gene selected from the group consisting of: CACNA1C, SLC1A1, ANK3, BDNF, and APOE; and an interpretive analysis of the neurophysiological significance of the patient's autonomic arousal system biomarker test results, the patient's emotional valence, attention, reward and executive brain function test results, and the patient's LTP-LTD axis, wherein the interpretive analysis addresses patient treatment, wherein the interpretive analysis comprises patient-specific information on response to a neurotherapeutic agent based on the biomarker test results; wherein the written description of the biomarker test results includes a visual representation of a brain region relevant to the biomarker; and a referral to a call center to receive additional interpretive information.

In one variation of an article of manufacture, the article is an interpretive neuropsychiatric report of patient-specific and dimensional information relevant to the treatment of a neuropsychiatric disorder, the article of manufacture comprising: a written description of a biomarker test result for a SLC6A4 (SERT) biomarker of the patient's autonomic arousal system axis, wherein the biomarker is related to a gene or a protein encoded or modulated by the SLC6A4 (SERT) gene; a written description of a biomarker test result specific to the patient for each of a COMT, SLC6A3, and DRD2 biomarker, related to the patient's emotional valence, attention, reward and executive brain function axis, wherein the biomarker is related to the COMT, SLC6A3, and DRD2 gene or a protein encoded or modulated by these genes; a written description of biomarker test results specific to the patient for at least the CACNA1C and ANK3 biomarkers, related to the patient's long-term potentiation and depression (LTP-LTD) function axis, the biomarker related to the CACNA1C and ANK3 gene or a protein encoded or modulated by the CACNA1C and ANK3 genes; an interpretive analysis of the neurophysiological significance of the patient's autonomic arousal system biomarker test results, the patient's emotional valence, attention, reward and executive brain function test results, and the patient's LTP-LTD axis, wherein the interpretive analysis addresses patient treatment, wherein the interpretive analysis comprises patient-specific information on response to a neurotherapeutic agent based on the biomarker test results; wherein the written description of the biomarker test results includes a visual representation of a brain region relevant to the biomarker; and a referral to a call center to receive additional interpretive information.

In some articles of the articles of manufacture described herein, the article may include a description of biomarker test results for biomarkers indicating dysfunction of metabolic (e.g., drug metabolism) including all of some of the following genes: MDR1, 5HT2C, MTHFR, CYP2D6, CYP2C19 and CYP3A5.

Part 2

Pharmaceutical compositions including one more ARB may be used to treat patients either with or without evidence of a dysfunction of the patient's angiotensin converting enzyme (and/or the hypothalamo-pituitary-adrenal (HPA) axis or autonomic arousal axis, generally).

In general, these pharmaceutical compositions may include one or more ARB (and particularly telmisartan) for the treatment of PTSD. The telmisartan may be used alone or preferably in combination with minocycline. The combination of telmisartan and minocycline has been found by the inventor to have surprising and synergistic effects; specifically the combination of the two may have beneficial therapeutic outcomes in PTSD that exceed those of either alone, or at levels that are much lower than those (therefore having much lower side-effects) than either alone.

As used in this application, except as otherwise expressly provided herein, each of the following terms shall have the meaning set forth below. Additional definitions are set forth throughout the application. The invention includes embodiments in which exactly one member of the group is present in, employed in, or otherwise relevant to a given product or method. The invention includes embodiments in which more than one, or all of the group members are present in, employed in, or otherwise relevant to a given product or method. Unless defined otherwise, all technical and scientific terms used herein have the same meaning as commonly understood by one of ordinary skill in the art to which this disclosure is related. For example, the definitions of different mental disorders can be found in Diagnostic and Statistical Manual of Mental Disorders, (DSM-). Treating, treatment, therapy: As used herein, the term “treating” or “treatment” or “therapy” refers to partially or completely alleviating, ameliorating, improving, relieving, delaying onset of, inhibiting progression of, reducing severity of, preventing relapse of, and/or reducing incidence of one or more symptoms or features of a disease, e.g., depression. For example, “treating” PTSD can refer to alleviating one or more symptoms of PTSD. Therapeutically effective amount: As used herein, the term a “therapeutically effective amount” of a drug is an amount effective to demonstrate a desired activity of the drug. For example, a therapeutically effective amount of ketamine is an amount effective for treating as described above. Dosage amount: The dosage amounts described herein are expressed in amounts of as a free base, and do not include the weight of a counter ion (e.g., sulfate) or any water or solvent molecules.

As used herein, the term “a PTSD means a patient either diagnosed with PTSD or who otherwise demonstrates symptoms associated with PTSD after experiencing or witnessing a traumatic event. The term “mild TBI” refers to the a patient who has had a traumatically induced physiological disruption of brain function, as manifested by at least one of the following: any period of loss of consciousness; any loss of memory for events immediately before or after the accident; any alteration in mental state at the time of the accident (e.g., feeling dazed, disoriented, or confused); and focal neurological deficit(s) that may or may not be transient; but where the severity of the injury does not exceed the following: loss of consciousness of approximately 30 minutes or less; after 30 minutes, an initial Glasgow Coma Scale (GCS) of 13-15; and posttraumatic amnesia (PTA) not greater than 24 hours.

The term a “pharmaceutically acceptable salt” refers to a pharmaceutically acceptable salt by addition of an organic or inorganic acid, or by addition of an organic or inorganic base. The acids include, but are not limited to, hydrochloric hydrobromic, hydroiodic acid, nitric, carbonic, sulfuric, phosphoric, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, 4-hydroxybenzoic, phenylacetic, mandelic, pamoic, methanesulfonic, ethanesulfonic, benzenesulfonic, pantothenic, 2-hydroxyethanesulfonic, toluenesulfonic, sulfanilic, cyclohexylaminosulfonic, stearic, alginic, beta-hydroxybutyric, galactaric or galacturonic acid. The bases include, but are not limited to, NaOH, KOH, LiOH, Ca(OH)2, Mg(OH)2, carbonates, bicarbonates triethylamine, benzylamine, diethanolamine, tert-butylamine, dicyclohexylamine, lysine, arginine, N,N′-dibenzylethylenediamine, choline, chloroprocaine, ethylenediamine, meglumine, or procaine; inorganic salts, e.g., sulfate, hydrochloride, and hydrobromide; and other salts which are currently in widespread pharmaceutical use and are listed in sources well known to those of skill in the art, such as The Merck Index. Any suitable constituent can be selected to make a salt of an active drug discussed herein, provided that it is non-toxic and does not substantially interfere with the desired activity. In addition to salts, pharmaceutically acceptable precursors and derivatives of the compounds can be employed.

telmisartan, e.g., 4′-[2-n-propyl-4-methyl-6-(1-methylbenzimidazol-2-yl)benzimidazol-1-ylme-thyl]biphenyl-2-carboxylic acid, has been developed for the treatment of hypertension and other medical indications as disclosed in EP 0 502 314 B1 and U.S. Pat. No. 5,591,762 and is already sold on the market under the trade name Micardis. It exists in two polymorphic forms as disclosed in WO 00/43370, U.S. Pat. No. 6,358,986 and U.S. Pat. No. 6,410,742. Sodium salts of telmisartan and its solvate, hydrate, and hemihydrate have been disclosed. The term “telmisartan” includes 4′-[2-n-propyl-4-methyl-6-(1-methylbenzimidazol-2-yl)benzimidazol-1-ylme-thyl]biphenyl-2-carboxylic acid in its neutral form as carboxylic acid as disclosed in EP 0 502 314 B1 and U.S. Pat. No. 5,591,762, or in one of its polymorphic forms as disclosed in WO 00/43370, U.S. Pat. No. 6,358,986 and U.S. Pat. No. 6,410,742, or in the form of a pharmaceutically acceptable salt or the solvate, hydrate, or hemihydrate thereof as disclosed in WO 03/037876, including but not limited to the sodium, potassium or ammonium salt. When a salt of telmisartan is used, the sodium salt may be preferred.

Among the ARBs are included candesartan eprosartan irbesartan, losartan olmesartan telmisartan and valsartan. Losartan and irbesartan have a greater affinity for cytochrome p450 (CYP) isoenzymes and, thus, are more likely to be implicated in drug interactions. Candesartan cilexetil, valsartan and eprosartan have variable but generally modest affinity and telmisartan has no affinity for any of the CYP isoenzymes.

As used herein, the term minocycline refers to the salts of minocycline useful in the present invention are the non-toxic acid addition salts, e.g. sulfonic, trichloroacetic, hydrochloric acid salts. The last named compound is also known as minocycline hydrochloride. Typically, minocycline hydrochloride is administered orally in a daily dosage of about 10 mg-1000 mg, 100 to about 400 mg, or a pharmaceutically acceptable salt, or an optical isomer, or a racemic mixture, or a pro-drug, or a solvate, or a crystalline form thereof.

In some embodiments, telmisartan can be co-administered with one or more other drugs that can ameliorate or exacerbate the symptoms of a neuropsychiatric disorder, including but are not limited to drugs include antidepressants such as lithium salts, carbamazepine, valproic acid, lysergic acid diethylamide (LSD), p-chlorophenylalanine, p-propylidopacetamide dithiocarbamate derivatives e.g., FLA 63; anti-anxiety drugs, e.g., diazepam; monoamine oxidase (MAO) inhibitors, e.g., iproniazid, clorgyline, phenelzine, tranylcypromine, and isocarboxazid; biogenic amine uptake blockers, e.g., tricyclic antidepressants such as desipramine, imipramine and amitriptyline; atypical antidepressants such as mirtazapine, nefazodone, bupropion; serotonin reuptake inhibitors e.g., fluoxetine, venlafaxine, and duloxetine; antipsychotic drugs such as phenothiazine derivatives (e.g., chlorpromazine (thorazine) and triflupromazine)), butyrophenones (e.g., haloperidol (Haldol)), thioxanthene derivatives (e.g., chlorprothixene), S and dibenzodiazepines (e.g., clozapine); benzodiazepines; dopaminergic agonists and antagonists e.g., L-DOPA, cocaine, amphetamine, a-methyl-tyrosine, reserpine, tetrabenazine, benztropine, pargyline; noradrenergic agonists and antagonists e.g., clonidine, phenoxybenzamine, phentolamine, tropolone.

The blood brain barrier (BBB) exists as a selective barrier formed by tight junctions between cerebral capillary endothelial cells, and is a critical regulator of brain homeostasis. In addition to the pharmacokinetic effects of drugs, the blood-brain barrier and its transporters have been implicated in CNS disorders such as PTSD and TBI, these transporters are not just bystanders, but rather active participants and thus potential targets for therapy. BBB damage that occur globally throughout the brain may be the main causes of non-impact, blast-induced brain injuries, including the spectrum of traumatic brain injury (TBI) and posttraumatic stress disorder (PTSD).

MDR1 modulation: A unique feature of brain endothelial cells is the existence of specific transport systems that regulate the entry of compounds necessary for brain metabolism, and chief among these are ATP-binding cassette (ABC) transporter, including an efflux pump also known as P-glycoprotein (P-gp) located on the BBB. This transmembrane protein is encoded by the MDR1 gene, and its normal physiological role is to protect the body from xenobiotic compounds by effluxing cytotoxic molecules for elimination. Upon ligand binding, basal transport activity of P-glycoprotein when activated will rapidly and reversibly reduce transport activity. Thus, xenobiotics are prevented from entering the sequestered milieu of the brain.

In addition to its physiological role, these transmembrane proteins also selectively regulate various pharmaceutical and nutraceutical compounds. It has been well established that ABC transporter expression on the luminal membrane of brain capillary endothelial cells is the major reason why it is such a challenge to deliver small molecule drugs to the brain. Moreover, numerous studies have shown that increased ABC transporter expression in the tissue leads to reduced drug accumulation in the brain and that decreased expression/activity leads to increased peripheral, rather than central drug accumulation. P-gp activity can be finely “tuned” by the appropriate modulators to make it more effective to selectively modulate the blood brain barrier in a desired manner to facilitate the transport of select drugs into the CNS. P-glycoprotein limits the oral availability of some drugs as well as their penetration into the brain. Emerging evidence suggests that P-gp may restrict the uptake of several antidepressants into the brain, thus contributing to the poor success rate of current antidepressant therapies. For example, P-gp restricts escitalopram transport across the blood-brain barrier (BBB). These studies further argue that the ability to control the activity/expression of this transporter in brain capillaries could be of substantial benefit in the treatment of a number of CNS diseases.

Disturbances in cortisol metabolism, which are part and parcel of conditions such as PTSD and TBI, may have an impact on the blood brain barrier and effect treatment outcomes. Cortisol acts as a P-gp substrate that reduces blood brain barrier permeability by inducing p-glycoprotein expression. In FKBP5 gene variants, reduced ligand-receptor binding may also alter normal g-protein kinetics and reduce efficacy of drugs across the blood brain barrier to treat PTSD.

Targeting elements of p-glycoprotein has the potential to improve drug delivery to the CNS and is an element of the current disclosure. Direct inhibition of P-gp to inhibit P-gp at the BBB can be used to increase the CNS targeted effects of certain drugs which otherwise would remain unrealized. P-gp modulators differ in their relative effect on the substrate specificity of P-gp, modifying the MDR phenotype. P-gp activity can be finely “tuned” by the appropriate modulators to make it more effective to selectively modulate the blood brain barrier in a desired manner to facilitate the transport of select drugs into the CNS. P-Glycoprotein substrates that inhibit MDR and potentially promote influx of the desired ARB to treat PTSD. Examples include antipsychotic drugs which are substrates of P-gp; quetipine, risperidone, olanzapine, and clozapine all have been shown to stimulate the ATPase activity of P-gp and reduce the efficiency of other desired drugs to cross the BBB efficiently.

MDR inhibitors may include telmisartan and minocycline, telmisartan has been identified as one of the most potent inhibitors of P-gp, telmisartan may thereby be a preferred ARB for use in CNS directed therapies as other ARBs failed to show interaction with P-gp, telmisartan, a nonpeptide angiotensin-II type 1-receptor antagonist, is an inhibitor of P-gp with an IC50-value of 0.38 μmol; telmisartan>(IC(50)=0.38+/−0.2 microM P-gp), cyclosporin A (Ki, 0.45 μmol/l) and quinidine (Ki, 0.82 μmol/l), telmisartan (2.4 μM) increased both uptake and permeability of verapamil in MDCK-II MDR1 cells.

A preferred MDR1 inhibitor may be minocycline. Minocycline is lipid soluble and has a high partition coefficient (39.4). Minocycline is a particularly suitable p-GP modulator to enhance delivery of ARB in the present invention. Minocycline increases the brain uptake of Riluzole in a 2.1-fold. Minocycline: (MNC) In mdr1a (−/−) and mdr1a (+/+) mice, mdr1a (−/−) mice showed higher brain uptake of minocycline than mdr1a (+/+).

The unique combination of these two agents is selected in a synergistic manner as to favorably alter the blood brain barrier.

Thus, in any of the compositions described herein, minocycline may be included. In addition to being an antibiotic, it has been suggested that minocycline may be helpful in inhibiting immune cell recruitment and/or activation. By itself, however, minocycline does not appear to be effective in treating PTSD.

As mentioned above, the stress response in PTSD is modulated by angiotensin receptor blockers. The hypothalamic-pituitary-adrenal (HPA) axis controls the physiological response to stress through the secretion of corticosteroid hormones such as cortisol. In response to stressors, cortisol is released, travels systemically through the blood and moves into the cytoplasm of cells cortisol binding involves a negative feedback mechanism that inhibits release of cortisol and prevents damaging effects of chronic activation.

An important regulator is the FK506 binding protein 51 (FKBP5). FKBP5 mediates an additional negative feedback loop on glucocorticoids. Glucocorticoids receptor (GR) activation results in rapid induction of FKBP5 which binds to the GR and decreases its ability to bind cortisol and to translocate to the nucleus. Thus, FKBP5 decreases systemic sensitivity to cortisol and reduces GR-mediated negative feedback modulation of the HPA axis. FKBP5 SNPs predicted hyperarousal/alterations in arousal and reactivity. The FKBP5 rs1360780 polymorphism is associated with plasma cortisol and PTSD. T carriers are similar to endophenotypes of people with post-traumatic stress disorder carrying the FKBP5 rs1360780 risk allele showed significant glucocorticoid resistance and loss of normal feedback inhibition compared with healthy controls. Angiotensin II (A-II) has a potentially important role in the control of cortisol secretion and 1 mediated through the AT1 receptor, stressful situations can giving rise to elevated levels of circulating angiotensin II ANG II modifies the expression level of transcription regulatory genes, increasing the expression of steroidogenic enzymes.

Angiotensin II plays a major role in both the exaggerated sympathetic, immune and hormonal response to stress, and conversely, blockade of angiotensin II (AT1) receptors may reduce the effects of stress. ANG II is thought to increase sympathetic outflow by promoting inflammation in the hypothalamus. ANG II has been shown to enhance Toll-like receptor4-mediated signaling on microglia. TLR4 and ANG II type 1 (AT1) receptor mRNA expression in hypothalamic microglia, providing molecular evidence for the potential interaction between these two receptors, evidence suggests that a-Ang II binding site is associated with activated microglia. AT1 Receptor antagonists-exert anti-anxiety effects in animal models. ARBs enhances retention of fear extinction suggesting that AT1 receptor antagonism may reduce fear memory.

In regards to their potential efficacy in TBI, Angiotensin II type 1 receptor blockers (ARBs) may ameliorate brain inflammation and reduce M1 microglia activation by promoting M2 polarization. This observation has led the inventor and others to disclose the use of ARBs for PTSD with or without co morbid TBI. Chronic restraint stress induces microglial activation, reflected in altered microglial morphology and immune factor expression. Acute and chronic restraint stress reduced the proportion of primed to ramified microglia and microglial CD40 expression. In ANG II induced microglial activation, can be attenuated by the microglial inhibitor minocycline.

CNS trauma may lead to an inflammatory response mediated by various components of the innate and adaptive immune system. The mechanisms whereby immune activation exacerbates CNS injury are many-fold. Immune cells resident to the CNS, such as astrocytes and microglia, are activated following CNS injury. Furthermore, peripheral immune cells are recruited to enter the CNS and also become activated. These cells may include monocytes/macrophages, neutrophils, and T lymphocytes. Recruitment and activation of these peripheral immune cells into the CNS after injury involves many of the same mechanisms by which these cells are recruited to and activated by injured tissue outside the CNS. Thus, inhibition of immune cell recruitment in response to CNS trauma is hypothesized to reduce the cellular dysfunction and death caused by these CNS insults.

The generation of new neurons within the hippocampus is mediated by proliferating neural stem/progenitor cells that are exquisitely sensitive to local signaling. Alterations in the brain microenvironment as a result of TBI may inhibit neurogenesis, leading to deficits in neurogenesis-dependent functions. Stress and the accompanying changes in stress hormones orchestrated by the hypothalamic-pituitary-adrenal (HPA) axis suppress hippocampal neurogenesis and lead to deficits in learning and memory. Glucocorticoids have played a central role in modeling this process as are pro-inflammatory cytokines and interleukin-1β (IL-1β), interleukin-6 and tumor necrosis factor-α (TNFα) which are also associated with brain trauma. Inflammation is also accompanied by the central production of pro-inflammatory cytokines. Among these are those which are found to be inhibitory to neurogenesis and reduce the likelihood of recovery from brain injury. The beneficial effects of inhibiting immune cell recruitment and/or activation in TBI may be achieved through the administration of minocycline.

Methods

The precise time of administration and amount of any particular composition that will yield the most effective treatment in a given patient may depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing. The amount of telmisartan and/or minocycline in a single dose formulation may vary depending upon the conditions to be treated, and the particular mode of administration. As a practical example germane to this disclosure; both PTSD and TBI have acute, sub-acute and chronic phases of the disorder. Thus, treatment parameters, including dosing and timing may vary accordingly.

Treatment may be initiated with smaller dosages which are less than the optimum dose of the compound. Thereafter, the dosage may be increased by small increments until the optimum therapeutic effect is attained.

The dosage of any compositions of the present invention may vary depending on the symptoms, age and body weight of the patient, the nature and severity of the disorder to be treated or prevented, the route of administration, and the form of the composition. Any of these compositions may be administered in a single dose or in divided doses. Dosages for the compositions of the present invention may be readily determined by techniques known to those of skill in the art or as taught herein.

In some embodiments, telmisartan-minocycline (TM) is administered once per day. In other embodiments, TM is administered multiple times per day, for example, two or three times per day. In some embodiments, the TM pharmaceutical composition of the present invention is a controlled release (or sustained release) composition. In some embodiments, controlled release (or sustained release) composition is a controlled release matrix tablet contains one or more release controlling polymers, such as cellulosic polymers, such as, but are not limited to, hydroxypropyl methylcellulose. More specifically, the one or more release controlling polymers may include a first hydroxypropyl methylcellulose having a viscosity of 80 to 120 cps (2% solution in water) and a second hydroxypropyl methylcellulose having a viscosity of 3,000 to 5,600 cps (2% solution in water). In some embodiments, the first hydroxypropyl methylcellulose and the second hydroxypropyl methylcellulose are present in a ratio of about 2:1 to about 4:1.

In some embodiments, the controlled release matrix tablet further includes a filler, such as, for example, microcrystalline cellulose. The tablet also may further include a lubricant, such as, for example, magnesium stearate. In some embodiments, the tablet also may further include colloidal silica. For a more detailed description of the controlled- or sustained-release systems, see e.g. U.S. Pat. Nos. 5,672,360; 5,968,551; 6,294,195; 7,270,831; and 7,514,100. The controlled release systems can also be prepared by methods known in the art (see e.g., Goodson, in Medical Applications of Controlled Release, vol. 2, pp. 115-138 (1984)). Other controlled release systems discussed in the review by Langer, Science 249:1527-1533 (1990) can be used as well.

In some embodiments, the TM pharmaceutical composition is a controlled release matrix tablet including a pharmaceutically effective amount of TM, particularly TM sodium salt, or its crystalline forms, and one or more release controlling polymers. The tablet may include about 10 mg, about 50 mg, about 100 mg, or about 200 mg TM, etc.

In some embodiments, the controlled release matrix tablet has a dissolution rate in vitro, when measured using a USP dissolution apparatus, type II (paddle) at 100 rpm in 900 mL simulated gastric fluid (pH about 1.2) at about 37° C., of less than 14% TM released after 1 hour, between 45% and 80% TM released after 7 hours, and greater than 90% TM released after 16 hours, by weight.

In some other embodiments, the controlled release matrix tablet has a dissolution rate in vitro, when measured using a USP dissolution apparatus, type II (paddle) at 100 rpm in 900 mL simulated gastric fluid (pH about 1.2) at about 37° C., of less than 20% TM released after 2 hours, between 50% and 80% TM released after 8 hours, and greater than 90% TM released after 14 hours, by weight.

In some embodiments, the controlled release matrix tablet, when orally administered to a patient, provides a median time to mean maximum plasma concentration (Tmax) of TM ranging from about 2.0 hours to about 4.0 hours, from about 2.5 hours to about 3.5 hours, or from 2.5 hours to about 3.0 hours.

Formulations

Minocycline and telmisartan may be administered by one or more of a variety of routes, including oral, intravenous, intramuscular, intra-arterial, intramedullary, intrathecal, subcutaneous, intraventricular, trans- or intra-dermal, interdermal, rectal, intravaginal, intraperitoneal, intracardiac, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

The present disclosure encompasses the delivery or administration of minocycline and/or telmisartan compositions described herein by any appropriate route taking into consideration likely advances in the sciences of drug delivery. In general, the most appropriate route of administration will depend upon a variety of factors including the nature of the composition (e.g., its stability in various bodily environments such as the bloodstream and gastrointestinal tract), the condition of the patient (e.g., whether the patient is able to tolerate particular routes of administration), etc.

These formulations may be prepared by conventional means, and, if desired, the compositions may be mixed with any conventional additives/excipients, including but are not limited to a binder, a disintegrating agent, a lubricant, a release control agent, a solubilizing agent, a suspension aid, an emulsifying agent, a coating agent, a sweetening agent, a flavoring agent, a perfuming agent, a colorant, a preservative or an antioxidant.

In any of the pharmaceutical composition formulations described herein, non-active agents, such as wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Compositions of the present invention may be suitable for oral, topical (including buccal and sublingual), rectal, vaginal, aerosol and/or parenteral administration. The formulations may conveniently be presented in unit dosage form and may be prepared by any methods well known in the art of pharmacy.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of an active ingredient. Compositions of the present invention may also be administered as a bolus, electuary, or paste.

In solid dosage forms for oral administration (capsules, tablets, pills, dragees, powders, granules and the like), the active agent(s) is/are mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, and patches.

For topical ocular administration compositions of this invention may take the form of solutions, gels, ointments, suspensions or solid inserts, formulated so that a unit dosage comprises a therapeutically effective amount of the active component or some multiple thereof in the case of a combination therapy.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

Also within the scope of the present disclosure are kits that comprise the pharmaceutical compositions (e.g., including minocycline and telmisartan) described herein. Kits typically include a label indicating the intended use of the contents of the kit and instructions for use. The term “label” includes any writing, or recorded material supplied on or with the kit, or which otherwise accompanies the kit. Accordingly, this disclosure provides a kit for treating a patient inflicted with depression, PTSD, and/or TBI, the kit may include: a dosage (e.g., ranging from about 0.1 to about 3.0 mg/kg body weight/day of minocycline and/or a dosage ranging from about 0.1 to about 10.0 mg/kg body weight/day of telmisartan; and instructions for using minocycline and telmisartan in any of the methods disclosed herein.

Described below are examples illustrating the use of the pharmaceutical compositions described herein.

### Example 1

### FKBP5 Assay

Single nucleotide determination: Genetic variation in the FKBP5 gene confers altered cortisol function and a poorly regulated neuroendocrine response to stress. A single nucleotide polymorphism (SNP) in FKBP5 (C to T SNP in intron 2, rs1360780) increases the ability of the GR to bind to the glucocorticoid response elements and induce FKBP5 expression. This “risk” T allele is associated with GR resistance and has been linked with PTSD, depressive and anxiety symptoms and disorders, and suicide.

FKBP5 Epigenetic assessment: Methylation of FKBP5. Methylation of the FKBP5 gene can also be contemplated as an element of the disclosure. Methylation of CPG islands of FKBP5 decreases the induction of FKBP5 that occurs following glucocorticoid binding to GR, and demethylation can be induced by prolonged glucocorticoid exposure. Sodium bisulfite modification of DNA using the EZ DNA methylation Kit and reverse primers can be used to amplify FKBP5 intron 7 as previously described. Percent DNA methylation at each CpG locus can be quantified with the PyroMark CpG.

MDR1 assessment: patient-to-patient variation in the effectiveness of CNS pharmacotherapy could be variable levels of blood-brain barrier efflux transporter induction by therapeutic drugs based upon genetic factors, such as MDR1, including the C3435T polymorphism of the MDR1 gene. The risk of drug resistance was significantly higher in patients bearing TT genotype in comparison to carriers of the homozygous CC genotype and minor allele carriers on rs2235040 and rs9282564 demonstrated significantly higher side effects, increased risk of switching and discontinuation.

A pre-clinical screening method to validate the effects of combining MDr1 substrates to ascertain their ability to increase the blood brain barrier permeability may include a flouroscein based assay to determine the IC50 of various MDR1 inhibitors to assess P-glycoprotein activity.

### Example 2

### Clinical Example

CLINICAL: a 34 year old war combat war veteran suffered symptoms of PTSD, including frequent nightmares, avoidance behavior and recurrent experiencing of the original trauma. His history was also remarkable for a blast injury due to an IED explosion that killed a member of his platoon. On return from active duty he was treated with several different antidepressants with no beneficial response. An MRI of the brain was completed which was reported as negative. He was moderately hypertensive and had chronic back problems and pain and sometimes used pain killers, including Percocet. He was prescribed telmisartan 40 mg and the dose was steadily increased to lower his blood pressure. PTSD symptoms did not seem to improve until the inventor added a low dose of minocycline, 100 mg daily. After 3 weeks on this regime, he noticed a decline in his anger and depression, as well as a reduction in headaches with improved concentration.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of treating a patient having post-traumatic stress disorder (PTSD), the method comprising:
determining that the patient has a polymorphism in the patient's angiotensin converting enzyme (ACE) gene;
treating the patient with an angiotensin receptor blocker (ARB).

2. The method of claim 1, wherein the angiotensin receptor blocker is losartan or telmisartan.

3. The method of claim 1, wherein treating the patient with an angiotensin receptor blocker comprises treating the patient with an angiotensin receptor blocker and a tetracycline.

4. The method of claim 1, wherein treating the patient with an angiotensin receptor blocker comprises treating the patient with an angiotensin receptor blocker (ARB) and a tetracycline, wherein the ARB comprises telmisartan and the tetracycline comprises minocycline.

5. The method of claim 1, wherein determining the patient has a polymorphism in the ACE gene comprises determining that the patient is CC homozygous in rs4311.

6. The method of claim 1, further comprising determining that the patient has a dysfunction in the FK506 binding protein (FKBP) gene.

7. The method of claim 1, further comprising determining the patient has a dysfunction in the Multi-Drug Resistance 1 (MDR1) gene.

8. The method of claim 1, wherein treating comprises giving the patient a single combination dose form comprises telmisartan between about 10-100 mg of telmisartan and about 10-400 mg of minocycline a day.

9. The method of claim 1, wherein treating comprises giving the patient a single combination dose form comprises telmisartan between about 10-100 mg of telmisartan and about 50-200 mg of minocycline a day.

10. A method of treating a patient having post-traumatic stress disorder (PTSD), the method comprising:
determining that the patient has a polymorphism in the patient's angiotensin converting enzyme (ACE) gene;
treating the patient with one or more of: an angiotensin-converting enzyme inhibitor and angiotensin receptor blocker if the polymorphism indicates a dysfunction in the ACE gene.

11. A method of treating a patient having post-traumatic stress disorder (PTSD), the method comprising:
delivering a first agent that targets the patient's hypothalamo-pituitary-adrenal (HPA) axis and down-regulates an effect of angiotensin in a subject to whom the composition is administered, wherein the first agent is telmisartan or a pharmaceutically acceptable salt thereof;
concurrently delivering a second agent that targets pro-inflammatory microglial states wherein the second agent is minocycline or a pharmaceutically acceptable salt thereof; and
wherein both the first agent and the second agent are delivered in amounts that are ineffective to treat PTSD when either the first agent or the second agent is administered alone.

12. The method of claim 11, wherein both the first agent and the second agent are delivered as part of a combination dose form comprising telmisartan and minocycline.

13. The method of claim 11, wherein both the first agent and the second agent are delivered as part of a combination dose form comprising telmisartan and minocycline, wherein the combination dose form comprises 10-100 mg of telmisartan and 10-400 mg of minocycline.

14. The method of claim 11, wherein both the first agent and the second agent are delivered as part of a combination dose form comprising telmisartan and minocycline, wherein the combination dose form comprises 10-100 mg of telmisartan and 50-200 mg of minocycline.

15. The method of claim 11, further comprising determining that the patient has a polymorphism in the angiotensin converting enzyme (ACE) gene prior to delivering the first agent and the second agent.

16. The method of claim 11, further comprising determining that the patient has a polymorphism in the ACE gene comprises determining that the patient is CC homozygous in rs4311 prior to delivering the first agent and the second agent.

17. The method of claim 11, further comprising determining that the patient has a dysfunction in the FK506 binding protein (FKBP) gene prior to delivering the first agent and the second agent.

18. The method of claim 11, further comprising determining the patient has a dysfunction in the Multi-Drug Resistance 1 (MDR1) gene prior to delivering the first agent and the second agent.

19. The method of claim 11, further comprising monitoring the patient's response to the treatment by measuring a biomarker.

20. A method of treating a patient having post-traumatic stress disorder (PTSD), the method comprising:
delivering a first agent that targets the patient's hypothalamo-pituitary-adrenal (HPA) axis and down-regulates an effect of angiotensin in a subject to whom the composition is administered, wherein the first agent is telmisartan or a pharmaceutically acceptable salt thereof;
concurrently delivering a second agent that targets pro-inflammatory microglial states wherein the second agent is minocycline or a pharmaceutically acceptable salt thereof; and
wherein both the first agent and the second agent are delivered as part of a combination dose form comprising telmisartan and minocycline in amounts that are ineffective to treat PTSD when either the first agent or the second agent is administered alone, wherein the combination dose form comprises 10-100 mg of telmisartan and 10-400 mg of minocycline.

21. A pharmaceutical composition for the treatment of post-traumatic stress disorder (PTSD) with or without co-morbid TBI, the active agents of the composition consisting essentially of: telmisartan or a pharmaceutically acceptable salt thereof, and minocycline or a pharmaceutically acceptable salt thereof.

22. The pharmaceutical composition of claim 21, having about 10-100 mg of telmisartan and about 10-400 mg of minocycline.

23. The pharmaceutical composition of claim 21, having about 10-100 mg of telmisartan and about 50-200 mg of minocycline.

24. The pharmaceutical composition of claim 21, including one or more additional non-active agents comprising a carrier or excipient, or a preservative.

25. A pharmaceutical composition for the treatment of post-traumatic stress disorder (PTSD), the composition comprising:
a first agent that targets the hypothalamo-pituitary-adrenal (HPA) axis and down-regulates an effect of angiotensin in a subject to whom the composition is administered, wherein the first agent is telmisartan or a pharmaceutically acceptable salt thereof; and
a second agent that targets pro-inflammatory microglial states wherein the second agent is minocycline or a pharmaceutically acceptable salt thereof,
wherein said pharmaceutical composition is a single combination dosage form, and
wherein both the first agent and the second agent are present within the composition in an amount that is ineffective to treat PTSD with or without co-morbid TBI when either the first agent or the second agent is administered alone because of reduced ability to overcome the blood brain barrier.

26. The pharmaceutical composition of claim 25, wherein the combination dose form comprises between about 10-100 mg of telmisartan and about 10-400 mg of minocycline.

27. The pharmaceutical composition of claim 25, wherein the combination dose form comprises between about 10-100 mg of telmisartan and about 50-200 mg of minocycline.

28. The pharmaceutical composition of claim 25, further comprising one or more additional agents, wherein the one or more additional agents comprises one or more of: a carrier, an excipient, a preservative, and a substance that increases the blood brain barrier permeability of one or more of the first agent and the second agent.

29. The pharmaceutical composition of claim 25, wherein the ratio of the amount of the first agent to the second agent is between about 100:1 and 1:1.

30. A pharmaceutical composition for the treatment of post-traumatic stress disorder (PTSD), the composition comprising:
a first agent that targets the hypothalamo-pituitary-adrenal (HPA) axis and down-regulates the effect of angiotensin in a subject to whom the composition is administered, wherein the first agent is telmisartan or a pharmaceutically acceptable salt thereof;
a second agent wherein the second agent is minocycline or a pharmaceutically acceptable salt thereof; and
one or more agents, wherein the one or more agents comprises a carrier or excipient, a preservative, or a substance that increases the blood brain barrier permeability of the first agent or the second agent,
wherein said pharmaceutical composition is a single combination dosage form, wherein both the first agent and the second agent are present within the composition in an amount that is ineffective to treat PTSD and/or TBI when either the first agent or the second agent is administered alone.
